(19) United States
(12) Patent Application Publication
(10) Pub. No.: US 2024/0041335 A1
(43) Pub. Date: Feb. 8, 2024

(54) DISPLAY DEVICE AND METHOD OF MEASURING BLOOD PRESSURE USING THE SAME

(71) Applicant: SAMSUNG DISPLAY CO., LTD., YONGIN-SI (KR)

(72) Inventor: Chul KIM, YONGIN-SI (KR)

(21) Appl. No.: 18/182,917

(22) Filed: Mar. 13, 2023

(30) Foreign Application Priority Data
Jul. 29, 2022 (KR) 10-2022-0094639

(51) Int. Cl. A61B 5/021; A61B 5/00

(52) U.S. Cl. CPC A61B 5/02116; A61B 5/7278; A61B 5/7203

(57) ABSTRACT

A display device includes a display panel including a plurality of pixels, a pressure sensor configured to sense pressure applied from outside of the display device, a photosensor configured to detect light, and a processor configured to receive a pressure signal sensed by the pressure sensor in each of a plurality of measurement segments, and a first pulse wave signal sensed by the photosensor in each of the plurality of measurement segments. The processor is further configured to identify at least one of the measurement segments as an abnormal measurement segment, remove a pulse waveform from the first pulse wave signal in the abnormal measurement segment, generate a pulse wave signal by interpolating the pulse waveform, and calculate blood pressure based on the pulse wave signal.

### CROSS-REFERENCE TO RELATED APPLICATIONS

This application claims priority under 35 U.S.C. § 119 to Korean Patent Application No. 10-2022-0094639, filed on Jul. 29, 2022 in the Korean Intellectual Property Office, the disclosure of which is incorporated by reference herein in its entirety.

### TECHNICAL FIELD

Embodiments of the disclosure relate to a display device, and a method of measuring blood pressure.

### DISCUSSION OF RELATED ART

A display device displays images and may be used in, for example, televisions, monitors, portable smartphones, tablet PCs, etc. A portable display device may have a variety of features in addition to displaying images. Examples of such features include camera functionality, fingerprint sensor functionality, etc.

As advances are made in the healthcare industry, methods for acquiring biometric information on health more conveniently are being developed including, for example, converting a conventional oscillometric blood pressure measurement device, which may include a light source, a sensor and a display, into a portable electronic device.

### SUMMARY

Aspects of the disclosure provide a display device that can recognize proximity touch as well as contact touch.

It should be noted that objects of the disclosure are not limited to the above-mentioned object, and other objects of the disclosure will be apparent to those skilled in the art from the following description.

According to an embodiment of the disclosure, pressure applied by a user is sensed with a pressure sensor in a display device, light reflected off a blood vessel of the user's finger or the like is sensed with a photosensor of a display panel, and a pulse wave signal according to the amount of the sensed light is analyzed, allowing the user's blood pressure to be measured.

According to an embodiment of the disclosure, a display device includes a display panel including a plurality of pixels, a pressure sensor configured to sense pressure applied from outside of the display device, a photosensor configured to detect light, and a processor configured to receive a pressure signal sensed by the pressure sensor in each of a plurality of measurement segments, and a first pulse wave signal sensed by the photosensor in each of the plurality of measurement segments. The processor is configured to identify at least one of the measurement segments as an abnormal measurement segment when a magnitude of the pressure signal sensed in the at least one of the measurement segments does not lie in a predetermined first threshold range, remove a pulse waveform from the first pulse wave signal in the abnormal measurement segment, generate a second pulse wave signal by interpolating the pulse waveform, calculate a third pulse wave signal having an amplitude of the second pulse wave signal based on the pressure signal sensed by the pressure sensor and the second pulse wave signal, and calculate blood pressure based on the third pulse wave signal.

In an embodiment, the first threshold range includes an upper limit pressure, a lower limit pressure, and a pressure width, and the pressure width is within 10 mmHg.

In an embodiment, the upper limit pressure and the lower limit pressure gradually increase in the plurality of measurement segments.

In an embodiment, the processor receives an additional pressure signal from the pressure sensor in each of the plurality of measurement segments when the magnitude of the pressure signal sensed in the at least one of the measurement segments does not lie within a predetermined second threshold range.

In an embodiment, the processor receives the first pulse wave signal from the photosensor a plurality of times.

In an embodiment, a pressure width of the second threshold range is greater than a pressure width of the first threshold range.

In an embodiment, the plurality of measurement segments includes first to nth measurement segments, the first pulse wave signal has one cycle in each of the first to n^{th} measurement segments, and n is a positive integer.

In an embodiment, when a kth measurement segment is the abnormal measurement segment, the processor generates the second pulse wave signal by calculating an average value of an amplitude of a (k−1)^{th} measurement segment and an amplitude of a (k+1)^{th} measurement segment as an amplitude of the pulse waveform, where k is a positive integer.

In an embodiment, the processor generates a peak detection signal based on an amplitude corresponding to a peak of each cycle of the third pulse wave signal.

In an embodiment, the processor calculates a peak value of the peak detection signal and a pressure value corresponding to the peak value of the peak detection signal, a diastolic blood pressure lower than the pressure value, a systolic blood pressure higher than the pressure value, and an average blood pressure depending on the pressure value.

In an embodiment, the processor calculates the pressure value corresponding to the peak value as the average blood pressure.

In an embodiment, a first pressure value smaller than a pressure value of about 60% to about 80% of the peak value in the peak detection signal and a second pressure value greater than the pressure value are calculated, and the first pressure value is calculated as a diastolic blood pressure and the second pressure value is calculated as a systolic blood pressure.

In an embodiment, each cycle of the first pulse wave signal includes a plurality of waveforms having different amplitudes, and a kth measurement segment is the abnormal measurement segment. The processor generates the second pulse wave signal by calculating an average value of a pulse waveform of a (k−1)th measurement segment and a pulse waveform of a (k+1)th measurement segment as the pulse waveform, where k is a positive integer.

In an embodiment, one cycle of the third pulse wave signal includes a plurality of waveforms having different amplitudes, and a reflected pulse wave ratio is calculated by:

$$RI = \frac{{R}_{P}}{{S}_{P}}$$

where a peak value of a first waveform among the plurality of waveforms is defined as a systolic pulse wave value, a peak value of a second waveform among the plurality of waveforms is defined as a reflected pulse wave value, the systolic pulse wave value is denoted by Sp, the reflected pulse wave value is denoted by Rp, and the reflected pulse wave ratio is denoted by RI.

In an embodiment, the reflected pulse wave ratio includes a first duration in which the reflected pulse wave ratio fluctuates within a first range, a second duration in which the reflected pulse wave ratio fluctuates within a second range, and a third duration in which the reflected pulse wave ratio fluctuates within a third range. A width of the first range and a width of the third range are less than a width of the second range.

In an embodiment, the processor is further configured to analyze the reflected pulse wave ratio to detect a start point of the second duration, calculate a first pressure value corresponding to the first pulse wave signal at a start time of the second duration, sets the first pressure value as a diastolic blood pressure, calculates a second pressure value corresponding to the first pulse wave signal at a start time of the third duration, and calculates the second pressure value as a systolic blood pressure.

According to an embodiment of the disclosure, a method of calculating a blood pressure by a display device includes sensing, by a pressure sensor configured to sense a pressure applied from outside of the display device, a pressure signal in each of first to nth measurement segments, and when a magnitude of the pressure signal sensed in one of the measurement segments does not lie within a predetermined first threshold range, identifying the measurement segment as an abnormal segment, where n is a positive integer. The method further includes sensing, by a photosensor configured to sense light, a first pulse wave signal having one cycle in each of the first to n^{th} measurement segments, and removing a pulse waveform of the first pulse wave signal in the abnormal measurement segment. The method further includes generating a second pulse wave signal by interpolating the pulse waveform, calculating a third pulse wave signal having an amplitude of the second pulse wave signal based on the pressure signal and the second pulse wave signal, and calculating the blood pressure based on the third pulse wave signal and indicating blood pressure information on a display panel of the display device.

In an embodiment, generating the second pulse wave signal by interpolating the pulse waveform includes, when a kth measurement segment is the abnormal measurement segment, generating the second pulse wave signal by calculating an average value of an amplitude of a (k−1)^{th} measurement segment and an amplitude of a (k+1)^{th} measurement segment as an amplitude of the pulse waveform. Herein, k is a positive integer.

In an embodiment, calculating the blood pressure based on the third pulse wave signal and indicating the blood pressure information on the display panel includes generating a peak detection signal based on an amplitude corresponding to a peak of each cycle of the third pulse wave signal, calculating a peak value of the peak detection signal and a pressure value corresponding to the peak value of the peak detection signal, and calculating a diastolic blood pressure lower than the pressure value, a systolic blood pressure higher than the pressure value, and an average blood pressure depending on the pressure value.

In an embodiment, the pressure value corresponding to the peak value is calculated as the average blood pressure.

According to embodiments, if the pressure applied by the user to the pressure sensor does not constantly increase, an inaccurate pulse wave signal may be obtained. When this happens, an abnormal measurement segment is extracted, and a pulse wave signal associated with the abnormal segment is removed and interpolated. As a result, the accuracy of blood pressure calculation may be increased.

It should be noted that effects of the disclosure are not limited to those described above, and other effects of the disclosure will be apparent to those skilled in the art from the following description.

### BRIEF DESCRIPTION OF THE DRAWINGS

The above and other aspects and features of the disclosure will become more apparent by describing in detail embodiments thereof with reference to the accompanying drawings, in which:

FIG. 1 is a plan view of a display device according to an embodiment of the disclosure.

FIG. 2 is a plan view of a display device according to an embodiment of the disclosure.

FIG. 3 is a block diagram showing a display device according to an embodiment of the disclosure.

FIG. 4 is a plan view showing a layout of pixels and photosensors of a display cell according to an embodiment of the disclosure.

FIG. 5 is a cross-sectional view taken along line I-I′ of FIG. 4 according to an embodiment of the disclosure.

FIG. 6 is a block diagram of a processor according to an embodiment of the disclosure.

FIG. 7 is a flowchart illustrating a method of measuring blood pressure according to an embodiment of the disclosure.

FIG. 8 is a graph of a pressure signal showing a pressure measurement value over time according to an embodiment of the disclosure.

FIGS. 9 to 11 are graphs of pulse wave signals showing a pulse wave measurement value over time according to an embodiment of the disclosure.

FIG. 12 is a flowchart illustrating a method of interpolating a first pulse wave signal according to an embodiment of the disclosure.

FIGS. 13 and 14 are graphs showing pulse wave signals according to an embodiment of the disclosure.

FIGS. 15 and 16 are graphs showing pulse wave signals according to an embodiment of the disclosure.

FIG. 17 is a flowchart illustrating a method of interpolating a first pulse wave signal according to an embodiment of the disclosure.

FIGS. 18 and 19 are graphs showing pulse wave signals according to an embodiment of the disclosure.

FIG. 20 is a flowchart illustrating a method of identifying an abnormal measurement segment by a display device according to yet an embodiment.

FIG. 21 is a graph showing a pulse wave signal according to yet an embodiment.

FIG. 22 is a graph showing a pulse wave signal and a slope sum function according to yet an embodiment.

FIG. 23 is a flowchart illustrating a method of calculating blood pressure according to an embodiment of the disclosure.

FIG. 24 is a graph showing a waveform of a peak detection signal according to an embodiment of the disclosure.

FIG. 25 is a flowchart illustrating a method of calculating blood pressure according to an embodiment of the disclosure.

FIG. 26 is a graph showing a waveform of one cycle of a pulse wave signal according to an embodiment of the disclosure.

FIG. 27 is a graph showing a pulse wave signal and a reflected pulse wave ratio according to an embodiment of the disclosure.

### DETAILED DESCRIPTION OF THE EMBODIMENTS

Embodiments of the present disclosure will be described more fully hereinafter with reference to the accompanying drawings. Like reference numerals may refer to like elements throughout the accompanying drawings.

It will be understood that when a component such as a film, a region, a layer, etc., is referred to as being “on”, “connected to”, “coupled to”, or “adjacent to” another component, it can be directly on, connected, coupled, or adjacent to the other component, or intervening components may be present. It will also be understood that when a component is referred to as being “between” two components, it can be the only component between the two components, or one or more intervening components may also be present. It will also be understood that when a component is referred to as “covering” another component, it can be the only component covering the other component, or one or more intervening components may also be covering the other component. Other words used to describe the relationships between components should be interpreted in a like fashion.

It will be understood that, although the terms “first,” “second,” etc. may be used herein to describe various elements, these elements should not be limited by these terms. These terms are only used to distinguish one element from another element. For example, a first element discussed below could be termed a second element in an embodiment. Similarly, the second element could also be termed the first element in an embodiment.

It should be understood that descriptions of features or aspects within each embodiment should typically be considered as available for other similar features or aspects in other embodiments, unless the context clearly indicates otherwise.

As used herein, the singular forms “a”, “an” and “the” are intended to include the plural forms as well, unless the context clearly indicates otherwise.

Herein, when two or more elements or values are described as being substantially the same as or about equal to each other, it is to be understood that the elements or values are identical to each other, the elements or values are equal to each other within a measurement error, or if measurably unequal, are close enough in value to be functionally equal to each other as would be understood by a person having ordinary skill in the art. For example, the term “about” as used herein is inclusive of the stated value and means within an acceptable range of deviation for the particular value as determined by one of ordinary skill in the art, considering the measurement in question and the error associated with measurement of the particular quantity (e.g., the limitations of the measurement system). For example, “about” may mean within one or more standard deviations as understood by one of the ordinary skill in the art. Further, it is to be understood that while parameters may be described herein as having “about” a certain value, according to exemplary embodiments, the parameter may be exactly the certain value or approximately the certain value within a measurement error as would be understood by a person having ordinary skill in the art. Other uses of these terms and similar terms to describe the relationships between components should be interpreted in a like fashion.

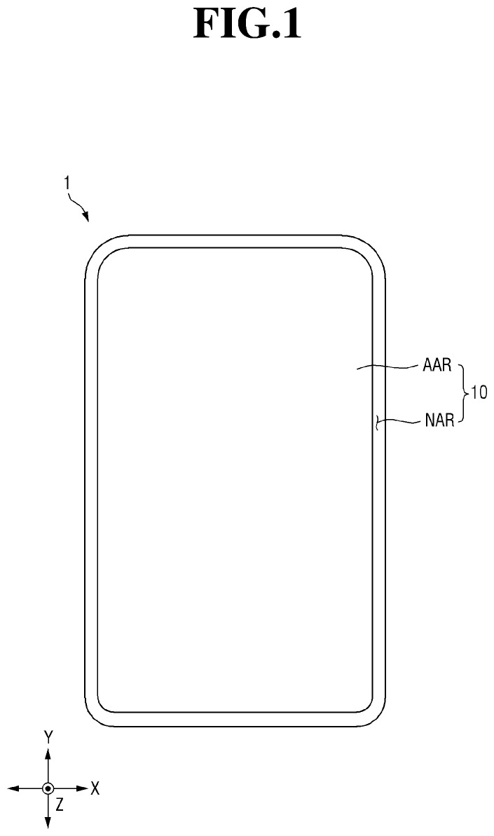

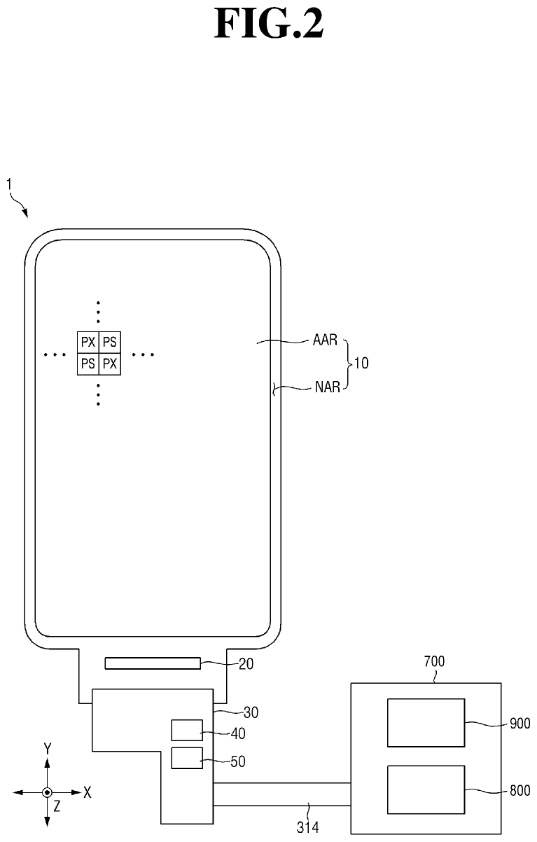

FIG. 1 is a plan view of a display device according to an embodiment of the disclosure. FIG. 2 is a plan view of a display device according to an embodiment of the disclosure.

Referring to FIGS. 1 and 2, a display device 1 may include a variety of electronic devices providing a display screen. Examples of the display device 1 include, but are not limited to, a mobile phone, a smartphone, a tablet PC, a mobile communications terminal, an electronic organizer, an e-book, a personal digital assistant (PDA), a portable multimedia player (PMP), a navigation device, an ultra mobile PC (UMPC), a television set, a game machine, a wristwatch-type electronic device, a head-mounted display, a personal computer monitor, a laptop computer, a vehicle instrument cluster, a digital camera, a camcorder, an outdoor billboard, an electronic billboard, various medical apparatuses, various inspection devices, various home appliances including a display area such as a refrigerator and a laundry machine, Internet of Things (IoT) devices, etc. Examples of the display device 1 to be described later include, but are not limited to, a smartphone, a tablet PC, a laptop computer, etc.

The display device 1 may include a display panel 10, a display driver 20, a circuit board 30, a pulse wave sensing circuit 50, a pressure-sensing circuit 40, a main circuit board 700, and a processor 800.

The display panel 10 may include an active area AAR and a non-active area NAR.

The active area AAR includes a display area where images are displayed. The active area AAR may completely overlap the display area. A plurality of pixels PX may be disposed in the display area for displaying images. Each of the pixels PX may include a light-emitting unit that emits light.

The active area AAR further includes a photo sensing area. The photo sensing area is a photosensitive area and senses the amount of incident light, the wavelength, etc. The photo sensing area may overlap the display area. According to an embodiment of the disclosure, the photo sensing area may completely overlap the active area AAR when viewed from the top (when viewed from a plan view). In this case, the photo sensing area may be identical to the display area. According to an embodiment, the photo sensing area may be disposed only in a part of the active area AAR. For example, the photo sensing area may be disposed only in a limited area necessary for fingerprint recognition. In this case, according to an embodiment, the photo sensing area may overlap a part of the display area DA but does not overlap another part of the display area.

A plurality of photosensors PS that responds to light may be disposed in the photo sensing area.

The non-active area NAR may surround the active area AAR. The display driver 20 may be disposed in the non-active area NAR. The display driver 20 may drive the plurality of pixels PX and/or the plurality of photosensors PS. The display driver 20 may output signals and voltages for driving the display panel 10. The display driver 20 may be implemented as an integrated circuit (IC) and may be mounted on the display panel 10. Signal lines for transferring signals between the display driver 20 and the active area AAR may be further disposed in the non-active area NAR. In an embodiment, the display driver 20 may be mounted on the circuit board 30.

The circuit board 30 may be attached to one end of the display panel 10 using, for example, an anisotropic conductive film (ACF). Lead lines of the circuit board 30 may be electrically connected to the pads of the display panel 10. The circuit board 30 may be, for example, a flexible printed circuit board (FPCB) or a flexible film such as a chip-on-film (COF).

The pulse wave sensing circuit 50 may be disposed on the circuit board 30. The pulse wave sensing circuit 50 may be implemented as an integrated circuit and may be attached to the upper surface of the circuit board 30. The pulse wave sensing circuit 50 may be connected to a display layer of the display panel 10. The pulse wave sensing circuit 50 may sense a photo current generated by photo charges incident on the plurality of photosensors PS of the display panel 10. The pulse wave sensing circuit 50 may recognize a user's pulse waves based on the photo current.

The pressure sensing circuit 40 may be disposed on the circuit board 30. The pressure sensing circuit 40 may be implemented as an integrated circuit and may be attached to the upper surface of the circuit board 30. The pressure sensing circuit 40 may be connected to the display layer of the display panel 10. The pressure sensing circuit 40 may sense an electrical signal by pressure applied to the plurality of pressure sensors of the display panel 10. The pressure sensing circuit 40 may generate pressure data according to a change in an electrical signal sensed by the pressure sensor and transmit the generated pressure data to the processor 800.

The main circuit board 700 may be, for example, a printed circuit board or a flexible printed circuit board.

The main circuit board 700 may include the processor 800.

The processor 800 may control all functions of the display device 1. For example, the processor 800 may output digital video data to the display driver 20 through the circuit board 30 so that the display panel 10 displays images. In addition, the processor 800 may receive touch data from a touch driver circuit to determine the coordinates of the user's touch, and then may execute an application indicated by the icon displayed at the coordinates of the user's touch.

The processor 800 may produce a pulse wave signal PPG reflecting changes in the blood according to heartbeats based on an optical signal input from the pulse wave sensing circuit 50. In addition, the processor 800 may calculate a user's touch pressure according to an electrical signal input from the pressure-sensing circuit 40. In addition, the processor 800 may calculate the user's blood pressure based on the pulse wave signal PPG and the pressure signal. In addition, the processor 800 may perform biometric authentication of the user based on the pulse wave signal PPG.

The processor 800 may be, for example, an application processor, a central processing unit, or a system chip implemented as an integrated circuit.

According to an embodiment, a mobile communications module capable of transmitting/receiving a radio signal to/from at least one of a base station, an external terminal and a server over a mobile communications network may be further mounted on the main circuit board 700. The wireless signal may include various types of data depending on, for example, a voice signal, a video call signal, or a text/multimedia message transmission/reception.

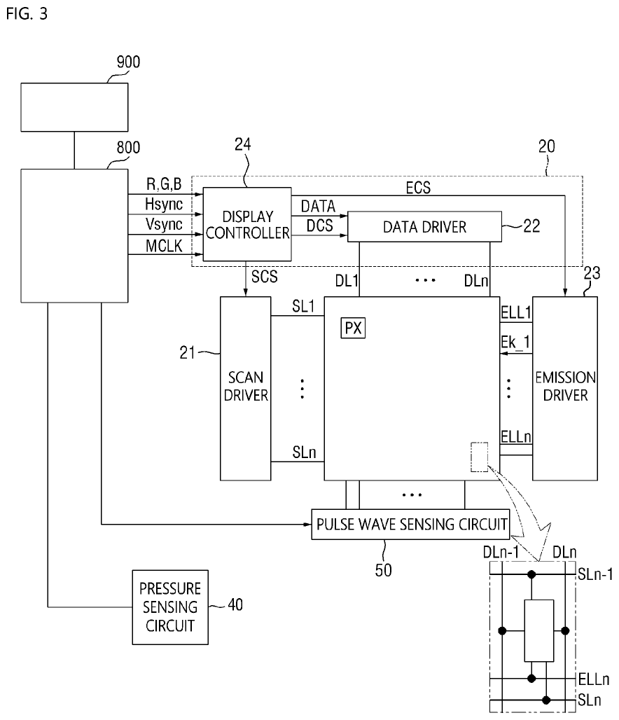

FIG. 3 is a block diagram showing a display device according to an embodiment of the disclosure.

Referring to FIG. 3, the display device 1 includes a display panel 10 including a plurality of pixels PX, a display driver 20, a scan driver 21, an emission driver 23, a pulse wave sensing circuit 50, a pressure sensing circuit 40, a processor 800 and a memory 900. Herein, n is a positive integer.

The processor 800 may receive electrical signals from the pulse wave sensing circuit 50 and the pressure sensing circuit 40 to yield the information on the user's blood pressure. The processor 800 may remove a segment of the pulse wave signal based on the received electrical signals and may interpolate the segment. The processor 800 may calculate a blood pressure based on the pulse wave signal and output the blood pressure information to the display panel 10.

The processor 800 drives and controls the pulse wave sensing circuit 50, the pressure-sensing circuit 40, and a display controller 24. The processor 800 may output image information to the display controller 24. For example, the processor 800 may output image information containing the pulse wave signal PPG, the blood-pressure measurement value, and the blood-pressure information thus obtained to the display controller 24. The display controller 24 may receive data and signals from the processor 800 including, for example, R, G, B image data, a horizontal sync signal Hsync, a vertical sync signal Vsync, and a signal MCLK.

The pulse wave sensing circuit 50 may sense a photo current generated by photo charges incident on the plurality of photosensors PS. The pulse wave sensing circuit 50 may generate a user's pulse wave signal based on the photo current.

The display controller 24 receives the image signal supplied from the processor 800. In addition, the display controller 24 may generate a scan control signal SCS for controlling the operation timing of the scan driver 21, an emission control signal ECS for controlling the operation timing of the emission driver 23, and a data control signal DCS for controlling the operation timing of the data driver 22. The display controller 24 may output the image data DATA and the data control signal DCS to the data driver 22. The display controller 24 may output the scan control signal SCS to the scan driver 21 and output the emission control signal ECS to the emission driver 23.

The display controller 24 may be electrically connected to the display panel 10 and/or the processor 800 via lines or may be connected over a communication network. According to an embodiment of the disclosure, at least a part of the display controller 24 may be attached directly to the display panel 10 in the form of a driving chip.

The data driver 22 may receive image data DATA and a data control signal DCS from the display controller 24. The data driver 22 may convert the image data DATA into an analog data voltage according to the data control signal DCS. The data driver 22 may output the converted analog data voltage to the data line DL in synchronization with the scan signal.

The scan driver 21 may generate scan signals according to the scan control signal SCS and may sequentially output the scan signals to the scan lines SL.

According to an embodiment, the display device 1 may further include driving voltage lines, common voltage lines, and supply voltage lines. The supply voltage lines may include a driving voltage line and a common voltage line. The driving voltage may be a high-level voltage for driving a light-emitting element and a photoelectric conversion element. The common voltage may be a low-level voltage for driving the light-emitting element and the photoelectric conversion element. In other words, the driving voltage may have a higher level than the common voltage. FIG. 3 illustrates, e.g., data lines DLn to DLn−1, and scan lines SLn to SLn−1.

Display control signals may include, for example, a scan control signal SCS, a data control signal DCS, and an emission control signal ECS. The display control signals may be output from the scan driver 21 and the data driver 22.

The emission driver 23 may generate an emission control Ek_1 in response to an emission control signal ECS and may sequentially output the emission signal Ek_1 to emission lines ELL1 to ELLn. Although the emission driver 23 is disposed separately from the scan driver 21 in the example shown in the drawings, it may be included in the scan driver 21 according to embodiments.

The data driver 22 and the display controller 24 may be included in the display driver 20 that controls the operation of the display panel 10. The data driver 22 and the display controller 24 may be implemented as an integrated circuit (IC) and mounted on the display driver 20.

Each of the plurality of pixels PX may be connected to at least one of the scan lines SL1 to SLn (where n is a positive integer), one of the data lines DL, and at least one of the emission lines ELL.

Each of the plurality of photosensors PS may be connected to one of the scan lines SL1 to SLn and one of lead-out lines.

The plurality of scan lines SL1 to SLn may connect the scan driver 21 with the plurality of pixels PX and the plurality of photosensors PS. The plurality of scan lines SL1 to SLn may provide scan signals output from the scan driver 21 to the plurality of pixels PX, respectively.

The plurality of data lines DL may c onnect the data driver 22 with the plurality of pixels PX, respectively. The plurality of data lines DL may provide image data output from the data driver 22 to the plurality of pixels PX, respectively.

The plurality of emission lines ELL may connect the emission driver 23 with the plurality of pixels PX, respectively. The plurality of emission lines ELL may provide emission control signals output from the emission driver 23 to the plurality of pixels PX, respectively.

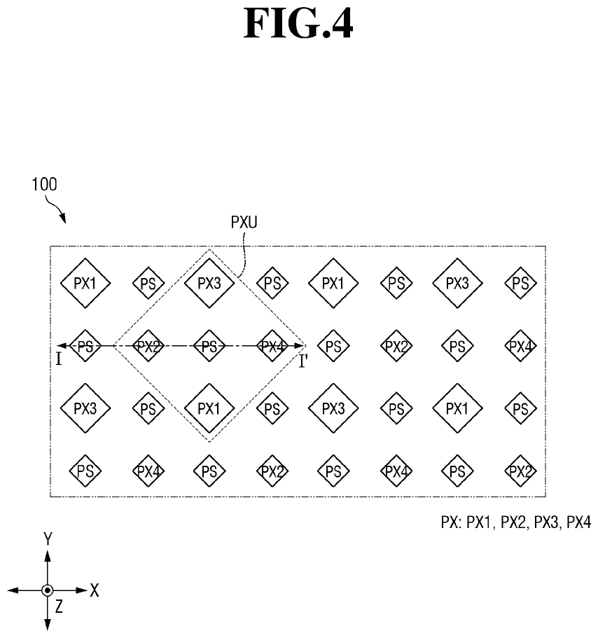

FIG. 4 is a plan view showing a layout of pixels and photosensors of a display cell according to an embodiment of the disclosure.

Referring to FIG. 4, a plurality of pixels PX and a plurality of photosensors PS may be repeatedly arranged in the display cell 100.

The plurality of pixels PX may include a first pixel PX1, a second pixel PX2, a third pixel PX3 and a fourth pixel PX4. For example, the first pixel PX1 may emit light of a red wavelength, the second pixel PX2 and the fourth pixel PX4 may emit light of a green wavelength, and the third pixel PX3 may emit light of a blue wavelength. Each of the pixels PX may include a plurality of emission areas that emit light. The plurality of photosensors PS may include a plurality of light-sensing areas in which incident light is sensed.

The first pixel PX1, the second pixel PX2, the third pixel PX3 and the fourth pixel PX4 and the photosensors PS may be arranged sequentially and repeatedly in a first direction X and a second direction Y. According to an embodiment of the disclosure, the first pixels PX1 and the third pixels PX3 may be alternately arranged in the first direction X to form a first row, while the second pixels PX2 and the fourth pixels PX4 may be alternately arranged in the first direction X to form a second row next to the first row. The pixels PX belonging to the first row may be arranged in a staggered manner in the first direction X with respect to the pixels PX belonging to the second row. The first row and the second row may be repeatedly arranged up to the n^{th} row.

Each of the photosensors PS may be disposed between the first pixels PX1 and the third pixels PX3 forming the first row and spaced apart therefrom. The first pixels PX1, the photosensors PS and the third pixels PX3 may be repeatedly and sequentially arranged along the first direction X. Each of the photosensors PS may be disposed between the second pixels PX2 and the fourth pixels PX4 forming the second row and spaced apart therefrom. The second pixels PX2, the photosensors PS and the fourth pixels PX4 may be repeatedly and sequentially arranged along the first direction X. The number of the photosensors PS in the first row may be equal to the number of the photosensors PS in the second row. The first row and the second row may be repeatedly arranged up to the n^{th} row.

As another example, in an embodiment, each of the photosensors PS may be disposed between the second pixel PX2 and the fourth pixel PX4 forming the second row, but are not disposed between the first pixel PX1 and the third pixel PX3 forming the first row. In other words, the photosensors PS are not disposed in the first row according to an embodiment.

Different pixels PX may have emission areas of different sizes. The sizes of the emission areas of the second pixels PX2 and the fourth pixels PX4 may be smaller than the sizes of the emission areas of the first pixels PX1 or the third pixels PX3. Although the shape of each of the pixels PX is a diamond in the example shown in the drawings, the shape of each of the pixels PX is not limited thereto, and may be, for example, a rectangle, an octagon, a circle, or other polygons.

A single pixel unit PXU may include a first pixel PX1, a second pixel PX2, a third pixel PX3 and a fourth pixel PX4. A pixel unit PXU refers to a group of color pixels capable of representing a grayscale level.

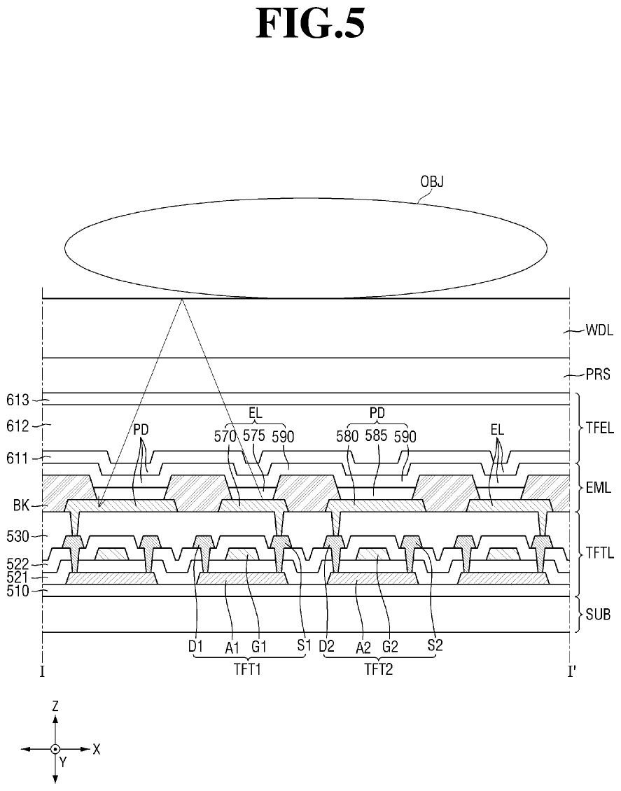

FIG. 5 is a cross-sectional view taken along line I-I′ of FIG. 4 according to an embodiment of the disclosure.

Referring to FIG. 5, a buffer layer 510 is disposed on a substrate SUB. The buffer layer 510 may include, for example, silicon nitride, silicon oxide, silicon oxynitride, or the like.

A first thin-film transistor TFT1 and a second thin-film transistor TFT2 may be disposed on the buffer layer 510.

The plurality of thin-film transistors TFT1 and TFT2 may respectively include semiconductor layers A1 and A2, a gate insulating layer 521 disposed on a part of the semiconductor layers A1 and A2, gate electrodes G1 and G2 disposed on the gate insulating layer 521, an interlayer dielectric film 522 covering the semiconductor layers A1 and A2 and the gate electrodes G1 and G2, and source electrodes S1 and S2 and drain electrodes D1 and D2 disposed on the interlayer dielectric film 522.

The semiconductor layers A1 and A2 may form channels of the first thin-film transistor TFT1 and the second thin-film transistor TFT2, respectively. The semiconductor layers A1 and A2 may include polycrystalline silicon. According to an embodiment, the semiconductor layers A1 and A2 may include, for example, monocrystalline silicon, low-temperature polycrystalline silicon, amorphous silicon, or an oxide semiconductor. The oxide semiconductor may include, for example, a binary compound (ABx), a ternary compound (ABxCy) and a quaternary compound (ABxCyDz) containing indium, zinc, gallium, tin, titanium, aluminum, hafnium (Hf), zirconium (Zr), magnesium (Mg), etc. Each of the semiconductor layers A1 and A2 may include a channel region, and a source region and a drain region doped with impurities.

The gate insulating layer 521 is disposed on the semiconductor layers A1 and A2. The gate insulating layer 521 electrically insulates the first gate electrode G1 from the first semiconductor layer A1 and electrically insulates the second gate electrode G2 from the second semiconductor layer A2. The gate insulating layer 521 may be made of an insulating material, for example, silicon oxide (SiOx), silicon nitride (SiNx), metal oxide, etc.

The first gate electrode G1 of the first thin-film transistor TFT1 and the second gate electrode G2 of the second thin-film transistor TFT2 are disposed on the gate insulating layer 521. The gate electrodes G1 and G2 may be formed above the channel regions of the semiconductor layers A1 and A2, respectively, such that they overlap the channel regions on the gate insulating layer 521.

The interlayer dielectric film may be disposed on the gate electrodes G1 and G2. The interlayer dielectric film 522 may include an inorganic insulating material such as, for example, silicon oxide (SiOx), silicon nitride (SiNx), silicon oxynitride, hafnium oxide and aluminum oxide. According to an embodiment, the interlayer dielectric film 522 may include a plurality of insulating films, and may further include a conductive layer forming a second electrode of a capacitor between the insulating films.

The source electrodes S1 and S2 and the drain electrodes D1 and D2 are disposed on the interlayer dielectric film 522. The first source electrode S1 of the first thin-film transistor TFT1 may be electrically connected to the drain region of the first semiconductor layer A1 through a contact hole penetrating the interlayer dielectric film 522 and the gate insulating layer 521. The second source electrode S2 of the second thin-film transistor TFT2 may be electrically connected to the drain region of the second semiconductor layer A2 through a contact hole penetrating the interlayer dielectric film 522 and the gate insulating layer 521. The source electrodes S1 and S2 and the drain electrodes D1 and D2 may include at least one metal including, for example, aluminum (Al), molybdenum (Mo), platinum (Pt), palladium (Pd), silver (Ag), magnesium (Mg), gold (Au), nickel (Ni), neodymium (Nd), iridium (Ir), chromium (Cr), calcium (Ca), titanium (Ti), tantalum (Ta), tungsten (W) and copper (Cu).

The planarization layer 530 may be formed on the interlayer dielectric film 522 and may cover the source electrodes S1 and S2 and the drain electrodes D1 and D2. The planarization layer 530 may be made of, for example, an organic insulating material. The planarization layer 530 may have a flat surface and may include contact holes exposing the source electrodes S1 and S2 and the drain electrodes D1 and D2, and may be included as part of a thin-film transistor layer TFTL.

An emission material layer EML may be disposed on the planarization layer 530. The emission material layer EML may include light-emitting elements EL, photoelectric conversion elements PD, and a bank layer BK. A light-emitting element EL may include a pixel electrode 570, an emissive layer 575, and a common electrode 590. A photoelectric conversion element PD may include a first electrode 580, a photoelectric conversion layer 585, and the common electrode 590.

The pixel electrode 570 of the light-emitting element EL may be disposed on the planarization layer 530. The pixel electrode 570 may be disposed in each pixel PX. The pixel electrode 570 may be connected to the first source electrode S1 or the first drain electrode D1 of the first thin-film transistor TFT1 through contact holes penetrating through the planarization layer 530.

The pixel electrode 570 of the light-emitting element EL may have, but is not limited to, a single-layer structure of molybdenum (Mo), titanium (Ti), copper (Cu) or aluminum (Al), or may have a stack of multiple films, e.g., a multi-layer structure of ITO/Mg, ITO/MgF, ITO/Ag and ITO/Ag/ITO including indium-tin-oxide (ITO), indium-zinc-oxide (IZO), zinc oxide (ZnO), indium oxide (In₂O₃), and silver (Ag), magnesium (Mg), aluminum (Al), platinum (Pt), lead (Pb), gold (Au) and nickel (Ni).

The first electrode 580 of the photoelectric conversion element PD may also be disposed on the planarization layer 530. The first electrode 580 may be disposed for each of the photosensors PS. The first electrode 580 may be connected to the second source electrode S2 or the second drain electrode D2 of the second thin-film transistor TFT2 through a contact hole penetrating through the planarization layer 530.

The first electrode 580 of the photoelectric conversion element PD may have, but is not limited to, a single-layer structure of molybdenum (Mo), titanium (Ti), copper (Cu) and aluminum (Al), or a multi-layer structure of ITO/Mg, ITO/MgF, ITO/Ag and ITO/Ag/ITO.

The bank layer BK may be disposed on the pixel electrode 570 and the first electrode 580. The bank layer BK may be formed where it overlaps the pixel electrode 570 to form an opening exposing the pixel electrode 570. The regions where the exposed pixel electrodes 570 and the emissive layer 575 overlap each other may be defined as the emission areas from which different lights are emitted in different pixels among PX1, PX2, PX3 and PX4.

In addition, the bank layer BK may be formed where it overlaps the first electrode 580 to form an opening exposing the first electrode 580. The opening exposing the first electrode 580 may provide a space in which the photoelectric conversion layer 585 of each of the photosensors PS is formed, and the area where the exposed first electrode 580 and the photoelectric conversion layer 585 overlap each other may be defined as a light-sensing area.

The bank layer BK may include an organic insulating material such as, for example, polyacrylate resin, epoxy resin, phenolic resin, polyamide resin, polyimide resin, unsaturated polyesters resin, poly phenylen ether resin, poly phenylene sulfide resin, and benzocyclobutene (BCB). As another example, the bank layer BK may include an inorganic material such as silicon nitride.

The emissive layer 575 may be disposed on the pixel electrode 570 of the light-emitting element EL exposed by the opening of the bank layer BK. The emissive layer 575 may include a high-molecular material or a low-molecular material, and may emit red, green and blue light from the pixels PX, respectively. The light emitted from the emissive layer 575 may contribute to image display or function as a light source incident on the photosensors PS. For example, a light source of a green wavelength emitted from the emission areas of the second pixel PX2 and the fourth pixel PX4 may work as a light source incident on the light-sensing area of the photosensor PS.

When the emissive layer 575 is formed of an organic material, a hole injecting layer and a hole transporting layer may be disposed under each emissive layer 575, and an electron injecting layer and an electron transporting layer may be disposed thereon. These layers may have a single-layer or multi-layer structure including an organic material.

The photoelectric conversion layer 585 may be disposed on the first electrode 580 of the photoelectric conversion element PD exposed by the opening of the bank layer BK. The regions where the exposed first electrode 580 and the photoelectric conversion layer 585 overlap each other may be defined as light-sensing areas of the photosensors PS. The photoelectric conversion layer 585 may generate photocharges in proportion to the incident light. The incident light may be light that was emitted from the emissive layer 575, was reflected and entered, or may be light provided from the outside of the display device irrespectively of the emissive layer 575. Charges generated and accumulated in the photoelectric conversion layer 585 may be converted into electrical signals utilized for sensing.

The photoelectric conversion layer 585 may include electron donors and electron acceptors. The electron donors may generate donor ions in response to light, and the electron acceptors may generate acceptor ions in response to light. When the photoelectric conversion layer 585 is formed of an organic material, the electron donors may include, but are not limited to, a compound such as, for example, subphthalocyanine (SubPc) and dibutylphosphate (DBP). The electron acceptors may include, but are not limited to, a compound such as, for example, fullerene, a fullerene derivative, and perylene diimide.

Alternatively, when the photoelectric conversion layer 585 is formed of an inorganic material, the photoelectric conversion element PD may be a p-n junction or pin-type phototransistor. For example, the photoelectric conversion layer 585 may have a structure in which an n-type semiconductor layer, an i-type semiconductor layer, and a p-type semiconductor layer are sequentially stacked on one another.

When the photoelectric conversion layer 585 is formed of an organic material, a hole injecting layer and a hole transporting layer may be disposed under each photoelectric conversion layer 585, and an electron injecting layer and an electron transporting layer may be disposed thereon. These layers may have a single-layer or multi-layer structure including an organic material.

The common electrode 590 may be disposed on the emissive layer 575, the photoelectric conversion layer 585 and the bank layer BK. The common electrode 590 may be disposed across the plurality of pixels PX and the photosensors PS such that it covers the emissive layer 575, the photoelectric conversion layer 585 and the bank layer BK. The common electrode 590 may include a conductive material having a low work function such as, for example, Li, Ca, LiF/Ca, LiF/Al, Al, Mg, Ag, Pt, Pd, Ni, Au, Nd, Ir, Cr, BaF, Ba, or a compound or mixture thereof, (e.g., a mixture of Ag and Mg). Alternatively, the common electrode 590 may include, for example, a transparent metal oxide, for example, indium-tin-oxide (ITO), indium-zinc-oxide (IZO), zinc oxide (ZnO), etc.

According to an embodiment, the common electrode 590 may be commonly disposed on the emissive layer 575 and the photoelectric conversion layer 585. In this case, the cathode electrode of the light-emitting element EL and the sensing cathode electrode of the photoelectric conversion element PD may be electrically connected. For example, the common voltage line connected to the cathode electrode of the light-emitting element EL may also be connected to the sensing cathode electrode of the photoelectric conversion element PD.

An encapsulation layer TFEL may be disposed on the emission material layer EML. The encapsulation layer TFEL may include at least one inorganic film, which may prevent or reduce permeation of oxygen or moisture into each of the emissive layer 575 and the photoelectric conversion layer 585. In addition, the encapsulation layer TFEL may include at least one organic film, which may protect the emissive layer 575 and the photoelectric conversion layer 585 from foreign particles such as, for example, dust. For example, the encapsulation layer TFEL may have a stack structure of a first inorganic film 611, an organic film 612 and a second inorganic film 613. The first inorganic film 611 and the second inorganic film 613 may be made up of multiple films in which one or more inorganic films of a silicon nitride layer, a silicon oxynitride layer, a silicon oxide layer, a titanium oxide layer and an aluminum oxide layer are alternately stacked on one another. The organic film 612 may be, for example, an acryl resin, an epoxy resin, a phenolic resin, a polyamide resin or a polyimide resin.

A pressure-sensing layer PRS may be disposed on the encapsulation layer TFEL. The pressure-sensing layer PRS may be disposed in the form of a panel or a film, and may be attached on the encapsulation layer TFEL by a bonding layer such as, for example, a pressure-sensitive adhesive (PSA) layer. Since the pressure-sensing layer PRS is located on paths of light emitted from the display layer 120, the pressure-sensing layer PRS may be transparent.

The pressure-sensing layer PRS may sense a pressure applied to the display device 1. When a user touches the upper surface of the display device 1, the pressing force of the touch input may be sensed by the pressure-sensing layer PRS. Pressure-sensing electrodes of the pressure-sensing layer PRS may be formed directly on the touch layer. In this case, the pressure-sensing layer PRS may be incorporated into the display panel 10 together with the display layer 120 and a touch layer.

A window WDL may be disposed on the pressure-sensing layer PRS. The window WDL may be disposed at the top of the display device 1 after display cells 100 undergo a cutting process and a module process, and may protect the elements of the display device 1. The window WDL may be made of, for example, glass or plastic.

FIG. 5 is a cross-sectional view showing the window WDL of the display device 1 on which a user's finger (or another object used for touch input) is placed. When a user's finger OBJ or the like comes into contact with the upper surface of the window WDL, lights output from the emission areas of pixels PX may be reflected off the user's finger OBJ or the like. The blood flow according to pressure in a blood vessel such as the user's finger OBJ may be different. Accordingly, the blood flow of the blood vessel such as the user's finger OBJ can be derived based on a difference in the amounts of reflected lights, e.g., lights incident on the photosensors PS. The blood pressure of the user OBJ can be measured through the photosensors PS and the pressure sensing layer PRS.

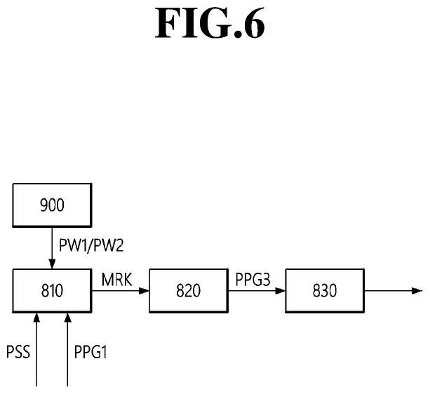

FIG. 6 is a block diagram of a processor according to an embodiment of the disclosure.

The processor 800 includes a measurement segment identifier 810, a pulse wave interpolator 820, and a blood pressure calculator 830.

The measurement segment identifier 810 may receive a pressure signal PSS having a pressure measurement value over time from the pressure sensing circuit 40. In addition, the measurement segment identifier 810 may receive data regarding a threshold range for determining an abnormal measurement segment from the memory 900. Accordingly, the measurement segment identifier 810 may identify an abnormal measurement segment from the pressure signal PSS. For example, the measurement segment identifier 810 may determine whether at least one of the a plurality of measurement segments of the pressure signal PSS (e.g., the kth measurement segment MRK) is an abnormal measurement segment. The measurement segment identifier 810 may output the identified abnormal measurement segment (e.g., the kth measurement segment MRK) to the pulse wave interpolator 820.

The memory 900 stores the information that is used to determine the abnormal measurement segment of the pressure signal PSS by the measurement segment identifier 810. For example, data regarding a first threshold range PW1 or a second threshold range PW2 of the pressure signal PSS may be stored in the memory 900. The memory 900 may output data used for determining an abnormal measurement segment to the measurement segment identifier 810.

The pulse wave interpolator 820 may receive a first pulse wave signal PPG1 having a pulse wave measurement value over time from the pulse wave sensing circuit 50. The pulse wave interpolator 820 may receive data regarding an abnormal measurement segment (e.g., the kth measurement segment MRK) from the measurement segment identifier 810. The pulse wave interpolator 820 may remove the pulse waveform of the first pulse wave signal PPG1 in the abnormal measurement segment. In addition, the pulse wave interpolator 820 may perform linear interpolation on the pulse waveform of the first pulse wave signal PPG1 in the abnormal measurement segment. It should be understood, however, that the disclosure is not limited thereto. The pulse wave interpolator 820 may perform Lagrangian interpolation or the like on the pulse waveform of the first pulse wave signal PPG1 in the abnormal measurement segment according to embodiments.

The pulse wave interpolator 820 may generate a second pulse wave signal PPG2 by interpolating the pulse waveform of the first pulse wave signal PPG1. The pulse wave interpolator 820 may generate a third pulse wave signal PPG3 having a magnitude of the pulse wave signal according to pressure based on the second pulse wave signal PPG2 and the pressure signal PSS.

In addition, the pulse wave interpolator 820 may remove noise from the third pulse wave signal PPG3 and/or increase the accuracy on the user's pulse wave. For example, the pulse wave interpolator 820 may calculate waveforms in at least two adjacent cycles among the cycles of the third pulse wave signal PPG3. The pulse wave interpolator 820 may generate a signal having the average value of the waveforms. In this manner, the accuracy on the user's pulse wave can be increased. The pulse wave interpolator 820 may output the third pulse wave signal PPG3 to the blood pressure calculator 830.

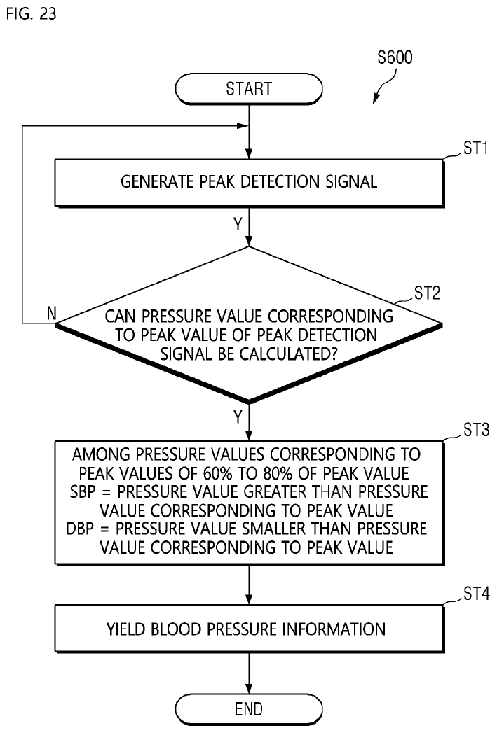

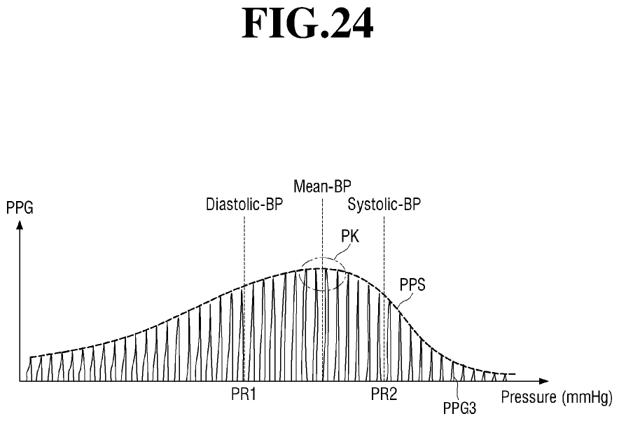

The blood pressure calculator 830 may generate a peak detection signal PPS (see FIG. 24) based on data on the cycle and amplitude of the pulse wave signal PPG. The blood pressure calculator 830 may calculate the blood pressure based on the peak value of the generated peak detection signal PPS. This will be described below with reference to FIG. 23.

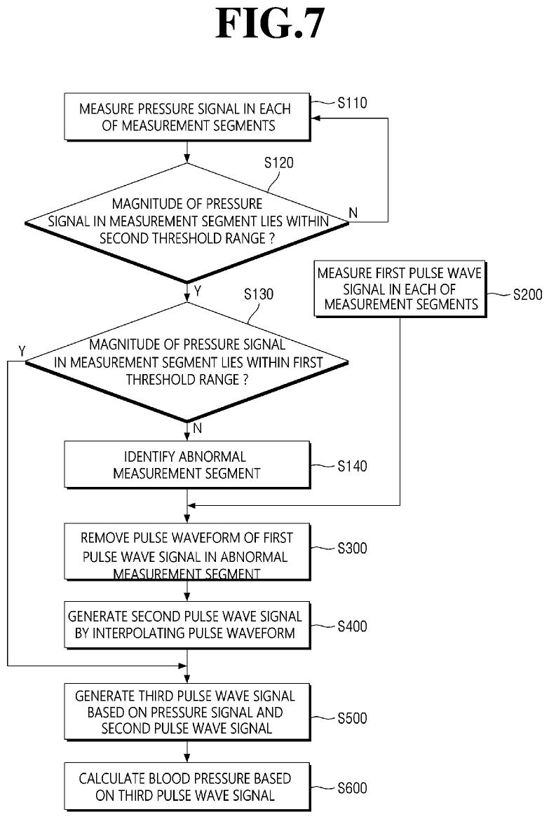

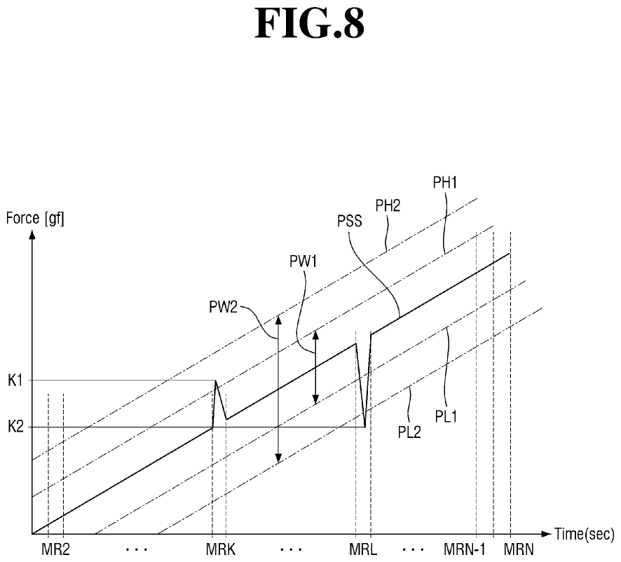

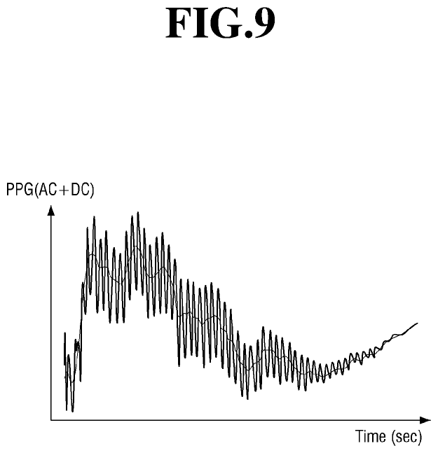

FIG. 7 is a flowchart illustrating a method of measuring blood pressure according to an embodiment of the disclosure. FIG. 8 is a graph of a pressure signal showing a pressure measurement value over time according to an embodiment of the disclosure. FIGS. 9 to 11 are graphs of pulse wave signals showing a pulse wave measurement value over time according to an embodiment of the disclosure. Hereinafter, a method of measuring blood pressure by the display device 1 will be described with reference to FIGS. 7 to 11.

Referring first to FIG. 7, a pressure sensing layer PRN measures a pressure signal PSS in each of the first to n^{th} measurement segments MR1 to MRN (operation S110).

Referring further to FIG. 8, a user may apply pressure to a position where a pressure sensor is disposed, and the pressure sensor disposed in the pressure sensing layer PRN may measure a pressure measurement value applied by the user. A method of generating a pulse wave signal will be described in detail. For example, while a user touches the display device 1 with a finger, the pressure measurement value measured by the pressure sensing layer PRN gradually increases over time and may reach a maximum value. As the pressure measurement value (e.g., contact pressure) increases, the blood vessel may constrict, and the blood flow rate may become small or zero.

Accordingly, the processor 800 may receive the pressure signal PSS having pressure measurement values in the first to n^{th} measurement segments MR1 to MRN generated by the pressure sensing circuit 40. Herein, each of the first to n^{th} measurement segments MR1 to MRN may be a subdivision for creating one cycle of the first pulse wave signal PPG1. Each of the first to n^{th} measurement segments MR1 to MRN may have a predetermined duration. For example, if one of the first to n^{th} measurement segments MR1 to MRN is defined as the kth measurement segment MRK, a predetermined duration of the kth measurement segment MRK may have one cycle of the first pulse wave signal PPG1. It should be understood, however, that the disclosure is not limited thereto. For example, according to embodiments, one measurement segment may have a larger duration or a smaller duration.

The magnitude of the pressure signal PSS received by the processor 800 may have different values in the first to n^{th} measurement segments MR1 to MRN. The magnitude of the pressure signal PSS may gradually increase from the first measurement segment MR1 to the n^{th} measurement segment MRN. For example, as shown in FIG. 8, the pressure signal PSS measured in the kth measurement segment MRK may have a first pressure value K1. In addition, the pressure signal PSS may have a second pressure value K2 in the l^{th} measurement segment MRL. The first pressure value K1 may be smaller than the second pressure value K2.

The pressure sensing circuit 40 that senses the pressure signal PSS functions in response to the user applying a gradually increasing pressure to the pressure sensing layer PRN. However, it may be difficult for the user to continuously apply a constantly increasing pressure to the pressure sensing layer PRN in all of the first to n^{th} measurement segments MR1 to MRN in which the user measures the blood pressure. Therefore, if the user applies a pressure out of the threshold range in at least one measurement segment, an inaccurate pressure signal PSS and pulse wave signal may be generated.

Subsequently, the processor 800 determines whether the magnitude of the pressure signal PSS in the measurement segments lies with the second threshold range PW2 (operation S120).

The second threshold range PW2 may have a second upper limit pressure PH2, a second lower limit pressure PL2, and a second pressure width. The second pressure width may be a pressure value between the second upper limit pressure PH2 and the second lower limit pressure PL2. In addition, the second upper limit pressure PH2, the second lower limit pressure PL2 and the second pressure width may gradually increase in the first to n^{th} measurement segments MR1 to MRN. For example, the second upper limit pressure PH2 in the kth measurement segment MRK may be smaller than the second upper limit pressure PH2 in the l^{th} measurement segment MRL.

Accordingly, the processor 800 may compare the magnitude of one of the first to n^{th} measurement segments MR1 to MRN with the second threshold range PW2. For example, as shown in FIG. 8, when the pressure signal PSS of the l^{th} measurement segment MRL has the second pressure value K2, the second pressure value K2 is greater than the second lower limit pressure PL2. In other words, the second pressure value K2 does not lie within the second threshold range PW2. Accordingly, when the magnitude of the pressure signal PSS of the l^{th} measurement segment MRL does not lie within the second threshold range PW2 (no in operation S120), the pressure sensing circuit 40 may measure the pressure signal PSS again in the first to n^{th} measurement segments MR1 to MRN. In addition, the pulse wave sensing circuit 50 may measure the first pulse wave signal PPG1 again in the first to n^{th} measurement segments MR1 to MRN.

On the other hand, when the pressure signal PSS of the kth measurement segment MRK has the first pressure value K1, the first pressure value K1 is less than the second upper limit pressure PH2. In other words, the first pressure value K1 lies within the second threshold range PW2. Accordingly, when the magnitude of the pressure signal PSS of the kth measurement segment MRK lies within the second threshold range PW2 (yes in operation S120), the processor 800 may determine whether the magnitude of the pressure signal PSS in the measurement segment lies within the first threshold range PW1 (operation S130).

The first threshold range PW1 may have a first upper limit pressure PH1, a first lower limit pressure PL1, and a first pressure width. The first pressure width may be a pressure value between the first upper limit pressure PH1 and the first lower limit pressure PL1. In addition, the first upper limit pressure PH1, the first lower limit pressure PL1 and the first pressure width may gradually increase from the first measurement segment MR1 to the n^{th} measurement segment MRN. For example, the first upper limit pressure PH1 in the kth measurement period MRK may be smaller than the first upper limit pressure PH1 in the l^{th} measurement period MRL. The first pressure width of the first threshold range PW1 may be smaller than the second pressure width of the second threshold range PW2. In addition, the first upper limit pressure PH1 of the first threshold range PW1 may be less than the second upper limit pressure PH2 of the second threshold range PW2. In addition, the first lower limit pressure PL1 of the first threshold range PW1 may be greater than the second lower limit pressure PL2 of the second threshold range PW2.

Accordingly, the processor 800 may compare the magnitude of one of the first to n^{th} measurement segments MR1 to MRN with the first threshold range PW1. For example, as shown in FIG. 8, when the pressure signal PSS of the kth measurement period MRK has the first pressure value K1, the first pressure value K1 is greater than the first upper limit pressure PH1. In other words, the first pressure value K1 does not lie within the first threshold range PW1. Therefore, when the magnitude of one of the first to n^{th} measurement segments MR1 to MRN does not lie within the first threshold range PW1, the processor 800 may identify the kth measurement segment MRK as an abnormal measurement segment (operation S140).

On the contrary, when the pressure signal PSS in each of the first to n^{th} measurement segments MR1 to MRN lies within the first threshold range PW1, the processor 800 may recognize the pressure signal PSS as the normal pressure signal PSS.

On the other hand, the pulse wave sensing circuit 50 measures the first pulse wave signal PPG1 in each of the first to n¹ measurement segments MR1 to MRN (operation S200).

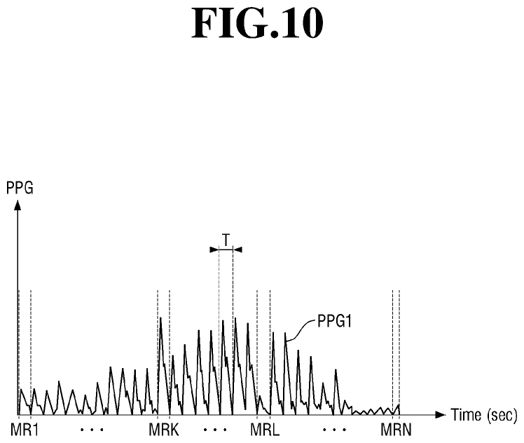

Referring to FIGS. 9 and 10, in order to generate the first pulse wave signal PPG1, the pulse wave information over time is also utilized along with the pressure data. During a systole of a heart, the blood ejected from the left ventricle of the heart moves to the peripheral tissues, and accordingly, the blood volume in the artery increases. In addition, red blood cells carry more oxygen in hemoglobin to the peripheral tissues during the systole of the heart. During a diastole of the heart, a part of the blood is sucked from the peripheral tissues towards the heart. At this time, when the light emitted from the display pixels is irradiated to the peripheral blood vessels, the irradiated light may be absorbed by the peripheral tissues. The light absorbance is dependent on the hematocrit ratio and the blood volume. The light absorbance may have the maximum value in the systole of the heart and the minimum value in the diastole of the heart. The light absorbance is in inverse proportion to the amount of light incident on the photosensors PS. Therefore, the light absorbance at a particular time point can be estimated based on data on the amount of received light incident on the photosensors PS2. In doing so, as shown in FIG. 9, the value of the first pulse wave signal PPG1 over time can be generated.

The pulse wave information over time reflects the maximum value of the light absorbance during the systolic phase of the heart and the minimum value of the light absorbance during the diastolic phase of the heart. In addition, the pulse wave information oscillates with every heartbeat cycle T. Accordingly, the pulse wave information may reflect a change in blood pressure according to heartbeats. Accordingly, the pulse wave sensing circuit 50 may measure the value of the first pulse wave signal PPG1 over the pressing time. The pulse wave signal PPG may include AC components as well as DC components. The processor 800 may remove the DC components from the pulse wave signal PPG and generate a first pulse wave signal PPG1 (see FIG. 10 where the graph is plotted with time on the x-axis and magnitude on the y-axis).

The processor 800 may receive the first pulse wave signal PPG1 generated from the pulse wave sensing circuit 50. The processor 800 may receive the first pulse wave signal PPG1 generated sequentially according to the first to n^{th} measurement segments MR1 to MRN. For example, if one of the first to n^{th} measurement segments MR1 to MRN is defined as a kth measurement segment MRK, the previous measurement segment adjacent to the kth measurement segment MRK is defined as the (k−1)^{th} measurement segment MR(K−1), and the next measurement segment adjacent to the kth measurement segment MRK is defined as the (k+1)^{th} measurement segment MR(K+1). Accordingly, the first pulse wave signal PPG1 may be sequentially generated in the (k−1)^{th} measurement segment MR(K−1), the kth measurement segment MRK, and the (k+1)^{th} measurement segment MR(K+1).

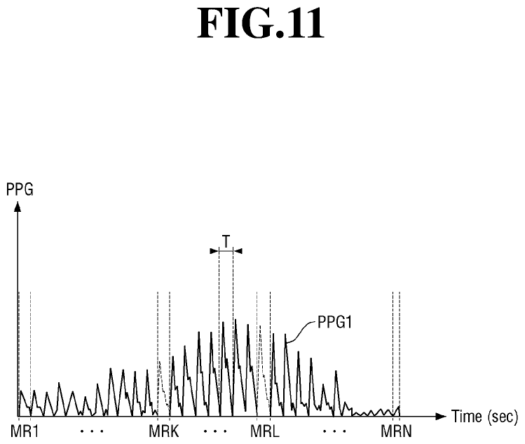

Subsequently, referring back to FIG. 7, the processor 800 removes the pulse waveform associated with the abnormal measurement segment from the first pulse wave signal PPG1 (operation S300). Referring further to FIG. 11, for example, the processor 800 may remove the pulse waveform of the first pulse wave signal PPG1 in the kth measurement segment MRK among the first to n^{th} measurement segments MR1 to MRN. Hereinafter, an example will be described where the pulse waveform is removed from the first pulse wave signal PPG1 in the kth measurement segment MRK.

Subsequently, the processor 800 interpolates the removed pulse waveform to generate a second pulse wave signal PPG2 (operation S400).

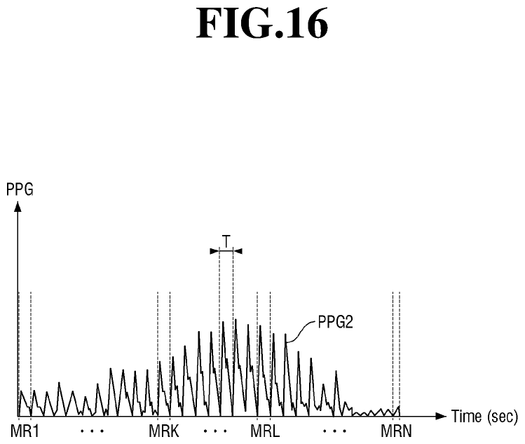

For example, the processor 800 may calculate the amplitudes of the pulse wave signals in the measurement segments adjacent to the abnormal measurement segment of the first pulse wave signal PPG1. In addition, the processor 800 may interpolate the first pulse wave signal PPG1 by calculating the average value of the amplitudes of the measurement segments adjacent to the abnormal measurement segment. Accordingly, the processor 800 may generate the second pulse wave signal PPG2 (see FIG. 16).

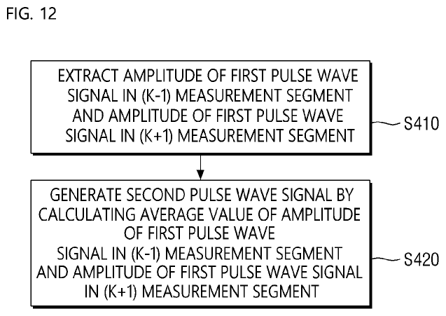

It is to be understood that the method of generating the second pulse wave signal PPG2 by interpolating the first pulse wave signal PPG1 by the processor 800 is not limited thereto. For example, according to embodiments, the processor 800 may generate the second pulse wave signal PPG2 (see FIG. 16) by perform linear interpolation or Lagrangian interpolation on the first pulse wave signal PPG1. This will be described below with reference to FIG. 12.

The processor 800 generates a third pulse wave signal PPG3 (see FIG. 24) based on the pressure signal PSS and the second pulse wave signal PPG2 (operation S500).

The processor 800 may generate the third pulse wave signal PPG3 (see FIG. 24) based on the pressure signal PSS input from the pressure sensing circuit 40 and the second pulse wave signal PPG2 input from the pulse wave sensing circuit 50. As described above, the pressure signal PSS is a signal having pressure measurement values in the first to n^{th} measurement segments MR1 to MRN, and the first pulse wave signal PPG1 is a signal indicative of data on the received light in the first to n^{th} measurement segments MR1 to MRN. Accordingly, the processor 800 may generate the third pulse wave signal PPG3 (see FIG. 24) having an amplitude of the pulse wave signal based on the pressure signal PSS and the second pulse wave signal PPG2.

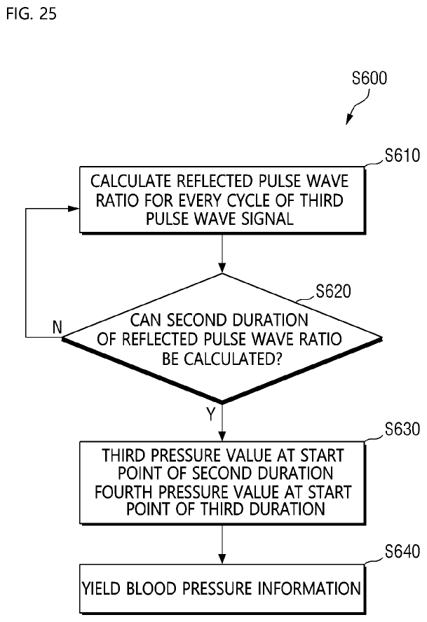

Lastly, the processor 800 calculates the blood pressure based on the third pulse wave signal PPG3 (operation S600). The processor 800 may generate a peak detection signal based on the peak values of the third pulse wave signal PPG3. In addition, the processor 800 may yield the user's blood pressure information based on a peak value of the peak detection signal. This will be described below with reference to FIG. 25.

In the method of measuring blood pressure of the display device 1 according to an embodiment, the display device 1 identifies an abnormal measurement segment, and removes and interpolates the pulse wave signal in the abnormal segment. In addition, since the display device 1 calculates the blood pressure based on the interpolated pulse wave signal, the blood pressure may be accurately measured.

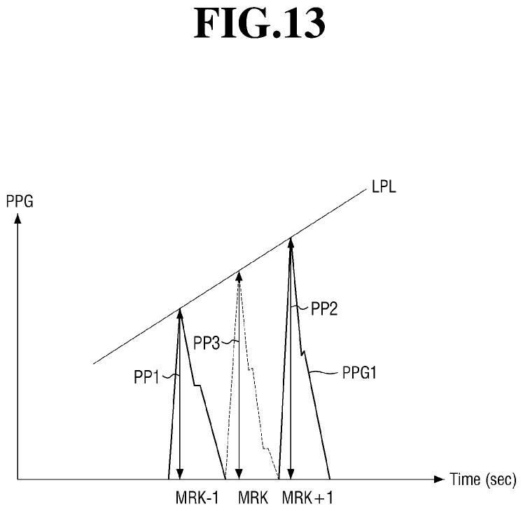

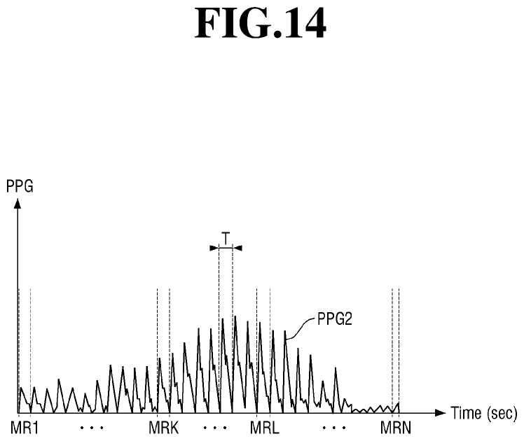

FIG. 12 is a flowchart illustrating a method of interpolating a first pulse wave signal according to an embodiment. FIGS. 13 and 14 are graphs showing pulse wave signals according to an embodiment of the disclosure. A method of interpolating a first pulse wave signal PPG1 will be described with reference to FIGS. 12 to 14.

First, the processor 800 extracts the amplitudes of the first pulse wave signal PPG1 in the (k−1)th measurement segment MR(K−1) and in the (k+1)^{th} measurement segment MR(K+1) (operation S410).

As described above, if one of the first to n^{th} measurement segments MR1 to MRN is defined as the kth measurement segment MRK, the previous measurement segment adjacent to the kth measurement segment MRK is defined as the (k−1)th measurement segment MR(K−1), and the subsequent measurement segment adjacent to the kth measurement segment MRK is defined as the (k+1)^{th} measurement segment MR(K+1). Hereinafter, an example will be described where the kth measurement segment MRK is identified as an abnormal measurement segment, and the pulse waveform of the first pulse wave signal PPG1 in the kth measurement segment MRK is removed.

Referring further to FIG. 13, the processor 800 may calculate a first amplitude PP1 of the first pulse wave signal PPG1 in the (k−1)^{th} measurement segment MR(K−1). For example, the (k−1)^{th} measurement segment MR(K−1) of the first pulse wave signal PPG1 may be one period of the first pulse wave signal PPG1. In this case, the first amplitude PP1 of the first pulse wave signal PPG1 in the (k−1)th measurement segment MR(K−1) may be the maximum value of the pulse waveform of the first pulse wave signal in the (k−1)th measurement segment MR(K−1). Alternatively, the first amplitude PP1 may be the peak value of the pulse waveform associated with the (k−1)th measurement segment MR(K−1) of the first pulse wave signal. As another example, when the pulse waveform associated with the (k−1)^{th} measurement segment MR(K−1) of the first pulse wave signal includes a plurality of peaks, the first amplitude PP1 may be the maximum value of the pulse waveform associated with the (k−1)th measurement segment MR(K−1) of the first pulse wave signal.

In addition, the processor 800 may calculate a second amplitude PP2 of the first pulse wave signal PPG1 in the (k+1)th measurement segment MR(K+1). The processor 800 may calculate the second amplitude PP2 of the first pulse wave signal PPG1 in the (k+1)^{th} measurement segment MR(K+1) in substantially the same manner as the first amplitude PP1 of the first pulse wave signal PPG1 in the (k−1)th measurement period MR(K−1). Thus, for convenience of explanation, a redundant description thereof will be omitted.

Subsequently, the processor 800 calculates the average value of the amplitude of the pulse wave signal in the (k−1)th measurement segment MR(K−1) and the amplitude of the pulse wave signal in the (k+1)th measurement segment MR(k+1) to generate a second pulse wave signal PPG2 (operation S420).

The processor 800 may calculate the average value of the pulse waveform in the (k−1)th measurement segment MR(K−1) and the pulse waveform in the (k+1)^{th} measurement segment MR(K+1). For example, the processor 800 may calculate the average value of the first amplitude PP1 of the first pulse wave signal PPG1 in the (k−1)th measurement segment MR(K−1) and the second amplitude PP2 of the first pulse wave signal PPG1 in the (k+1)^{th} measurement segment as a third amplitude PP3 in the kth measurement segment MRK.

In addition, the processor 800 may generate a linear correction function LPL that has the first amplitude PP1 in the (k−1)th measurement segment MR(K−1) and the second amplitude PP2 in the (k+1)th measurement segment MR(K+1), and may generate a second pulse wave signal PPG2 so that it is in contact with the linear correction function LPL in the kth measurement segment MRK. In this case, the third amplitude PP3 in the kth measurement segment MRK may be the average value of the first amplitude PP1 and the second amplitude PP2.

It should be understood, however, that the disclosure is not limited thereto. For example, according to embodiments, the third amplitude PP3 of the kth measurement segment MRK may be calculated using the average value of other segments that are not adjacent to the kth measurement segment MRK. For example, the processor 800 may generate a linear correction function LPL having the amplitude of the (k−2)th measurement segment and the amplitude of the (k+1)th measurement segment MR(K+1), and may calculate the third amplitude PP3 in the kth measurement segment MRK so that it is in contact with the linear correction function LPL in the kth measurement segment MRK. Accordingly, referring further to FIG. 14, the processor 800 may interpolate the removed pulse waveform of the first pulse wave signal PPG1 in the abnormal measurement segment. For example, the second pulse wave signal PPG2 having the magnitude of the signal in each of the first to n^{th} measurement segments MR1 to MRN may be generated.

In summary, the processor 800 generates the second pulse wave signal PPG2 by interpolating the removed pulse waveform of the first pulse wave signal PPG1 in the abnormal measurement segment. For example, the processor 800 may generate the second pulse wave signal PPG2 by correcting the signal of the abnormal measurement segment in the first pulse wave signal PPG1. Accordingly, when the display device 1 calculates the blood pressure based on the pulse wave signal, an accurate blood pressure can be calculated by correcting the abnormal pulse waveform.

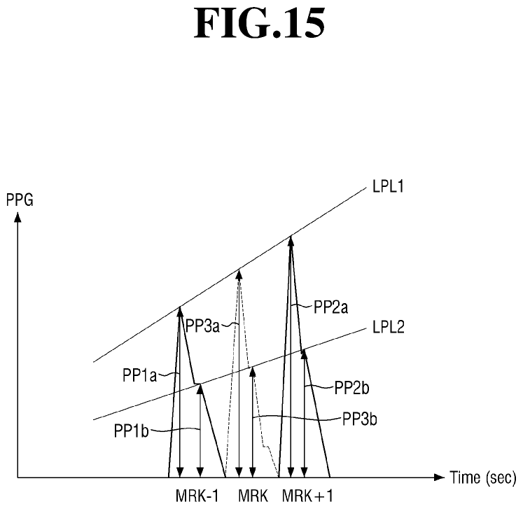

FIGS. 15 and 16 are graphs showing pulse wave signals according to an embodiment of the disclosure.

An embodiment of FIGS. 15 and 16 is substantially identical to an embodiment of FIGS. 12 to 14 except that a processor 800 calculates a first sub-amplitude PP1b and a second sub-amplitude PP2b. Thus, for convenience of explanation, a redundant description thereof will be omitted.

The processor 800 extracts the pulse waveforms of the first pulse wave signal PPG1 in the (k−1)th measurement segment MR(K−1) and in the (k+1)^{th} measurement segment MR(K+1). Subsequently, the processor 800 calculates the average value of the waveform of the pulse wave signal in the (k−1)^{th} measurement segment MR(K−1) and the waveform of the pulse wave signal in the (k+1)^{th} measurement segment MR(k+1) to generate a second pulse wave signal PPG2.

For example, referring to FIGS. 15 and 16, the processor 800 may calculate a pulse waveform of the (k−1)^{th} measurement segment MR(K−1). For example, the processor 800 may calculate a first main amplitude PP1a and a first sub-amplitude PP1b of the (k−1)^{th} measurement segment MR(K−1). In this case, each of the first to n^{th} measurement segments MR1 to MRN of the first pulse wave signal PPG1 may correspond to one period of the first pulse wave signal PPG1. In addition, one of the measurement segments of the first pulse wave signal PPG1 may include a waveform having a plurality of amplitudes. The first main amplitude PP1a is defined as the peak value of the waveform having the greatest value among the plurality of waveforms, and the first sub-amplitude PP1b is defined as the peak value of the waveform having the second greatest value among the plurality of waveforms. In addition, the processor 800 may calculate a pulse waveform in the (k+1)^{th} measurement segment MR(K+1). For example, the processor 800 may calculate a second main amplitude PP2a and a second sub-amplitude PP2b of the (k+1)th measurement period MR(K+1).

Accordingly, the processor 800 may calculate the average value of the first main amplitude PP1a and the second main amplitude PP2a in the kth measurement segment MRK as a third main amplitude PP3a of the kth measurement segment MRK. In addition, the processor 800 may calculate the average value of the first sub-amplitude PP1b and the second sub-amplitude PP2b in the kth measurement segment MRK as a third sub-amplitude PP3b of the kth measurement segment MRK. Accordingly, the processor 800 may generate a pulse waveform in the kth measurement segment MRK. The processor 800 may generate a second pulse wave signal PPG2 as in the example of FIG. 16 based on the generated pulse waveform of the kth measurement segment MRK.

In addition, the processor 800 may generate a first linear correction function LPL1 that has the first main amplitude PP1a in the (k−1)th measurement segment MR(K−1) and the second main amplitude PP2a in the (k+1)^{th} measurement segment MR(K+1). In addition, the processor 800 may generate a second linear correction function LPL2 that has the first sub-amplitude PP1b in the (k−1)th measurement segment MR(K−1) and the second sub-amplitude PP2b in the (k+1)th measurement segment MR(K+1). The processor 800 may calculate a third main amplitude PP3a in the kth measurement segment MRK so that it is in contact with the first linear correction function LPL1. In addition, the processor 800 may calculate a third sub-amplitude PP3b in the kth measurement segment MRK so that it is in contact with the second linear correction function LPL2 in the kth measurement segment MRK. Accordingly, the processor 800 may generate a pulse waveform in the kth measurement segment MRK. The processor 800 may generate a second pulse wave signal PPG2 as in the example of FIG. 16 based on the generated pulse waveform of the kth measurement segment MRK.

According to an embodiment, the processor 800 generates the second pulse wave signal PPG2 by interpolating the pulse waveform removed in the abnormal measurement segment of the first pulse wave signal PPG1. For example, the processor 800 may generate the second pulse wave signal PPG2 by correcting the signal of the abnormal measurement segment in the first pulse wave signal PPG1. Accordingly, when the display device 1 calculates the blood pressure based on the pulse wave signal, the accurate blood pressure can be calculated by correcting the abnormal pulse waveform.

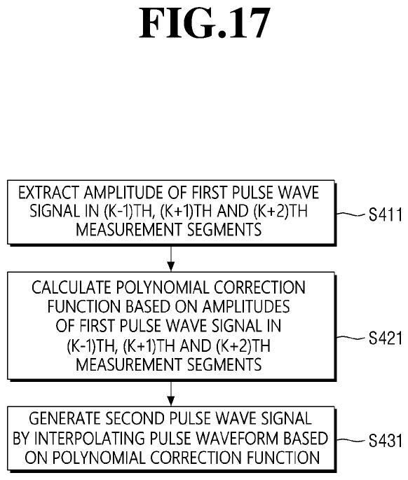

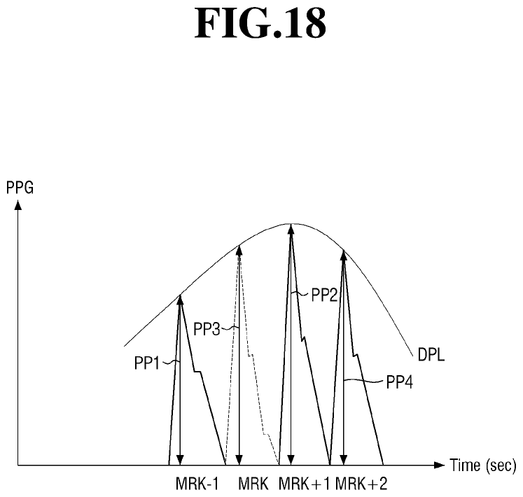

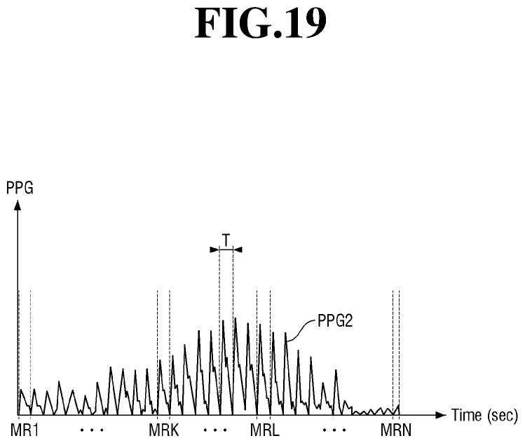

FIG. 17 is a flowchart illustrating a method of interpolating a first pulse wave signal according to an embodiment of the disclosure. FIGS. 18 and 19 are graphs showing pulse wave signals according to an embodiment of the disclosure.

An embodiment of FIGS. 17 to 19 is substantially identical to an embodiment of FIGS. 12 to 14 except that a processor 800 calculates a polynomial correction function DPL. Thus, for convenience of explanation, a redundant description thereof will be omitted.

Referring to FIG. 17, first, the processor 800 extracts the amplitudes of a first pulse wave signal PPG1 in the (k−1)th measurement segment MR(K−1), in the (k+1)^{th} measurement segment MR(K+1), and in the (k+2)th measurement segment MR(K+2) (operation S411).

As described above, if one of the first to n^{th} measurement segments MR1 to MRN is defined as the kth measurement segment MRK, the previous measurement segment adjacent to the kth measurement segment MRK is defined as the (k−1)¹ measurement segment MR(K−1), the subsequent measurement segment adjacent to the kth measurement segment MRK is defined as the (k+1)^{th} measurement segment MR(K+1), and the subsequent measurement segment adjacent to the (k+1)^{th} measurement segment MR(K+1) is defined as the (k+2)^{th} measurement segment MR(K+2). Hereinafter, an example will be described where the kth measurement segment MRK is identified as an abnormal measurement segment, and the pulse waveform of the first pulse wave signal PPG1 in the kth measurement segment MRK is removed.

Referring to FIG. 18, the processor 800 may calculate a first amplitude PP1 of the first pulse wave signal PPG1 in the (k−1)^{th} measurement segment MR(K−1). For example, the (k−1)^{th} measurement segment MR(K−1) of the first pulse wave signal PPG1 may be one period of the first pulse wave signal PPG1. In this case, the first amplitude PP1 of the first pulse wave signal PPG1 in the (k−1)^{th} measurement segment MR(K−1) may be the maximum value of the pulse waveform of the first pulse wave signal in the (k−1)^{th} measurement segment MR(K−1). Alternatively, the first amplitude PP1 may be the peak value of the pulse waveform associated with the (k−1)th measurement segment MR(K−1) of the first pulse wave signal. As another example, when the pulse waveform associated with the (k−1)^{th} measurement segment MR(K−1) of the first pulse wave signal includes a plurality of peaks, the first amplitude PP1 may be the maximum value of the pulse waveform associated with the (k−1)^{th} measurement segment MR(K−1) of the first pulse wave signal. In addition, the processor 800 may calculate a second amplitude PP2 of the (k+1)^{th} measurement segment MR(K+1) and a fourth amplitude PP4 of the (k+2)th measurement segment MR(K+2). A method of calculating the second amplitude PP2 of the (k+1)¹ measurement segment MR(k+1) and the fourth amplitude PP4 of the (k+2)^{th} measurement segment MR(K+2) is substantially identical to the method of calculating the first amplitude PP1 of the (k−1)^{th} measurement segment MR(K−1). Thus, for convenience of explanation, a redundant description thereof will be omitted.

Subsequently, the processor 800 calculates the polynomial correction function DPL based on the amplitudes of the pulse wave signals in the (k−1)^{th}, the (k+1)th and the (k+2)^{th} measurement segments MRK−1, MR(K+1) and MR(K+2) (operation S421).

Referring to FIGS. 18 and 19, the processor 800 may calculate the polynomial correction function DPL that has the first amplitude PP1 in the (k−1)^{th} measurement segment MR(K−1), has the second amplitude PP2 in the (k+1)^{th} measurement segment MR(K+1), and has the fourth amplitude PP4 in the (k+2)th measurement segment MR(K+2). For example, the polynomial correction function DPL may come into contact with the first pulse wave signal PPG1 in the (k−1)^{th}, the (k+1)^{th} and the (k+2)^{th} measurement segments MR(K−1), MR(K+1) and MR(K+2). In this case, the polynomial correction function DPL may be a polynomial having an order of 2 or more.

Accordingly, the processor 800 may generate the second pulse wave signal PPG2 by interpolating the pulse waveform based on the polynomial correction function DPL (operation S431). For example, the processor 800 may calculate a third amplitude PP3 of the kth measurement segment MRK so that it comes into contact with the polynomial correction function DPL in the kth measurement segment MRK. The processor 800 may generate a pulse waveform based on the amplitude in the kth measurement segment MRK. The processor 800 may generate a second pulse wave signal PPG2 as in the example of FIG. 19 based on the generated pulse waveform of the kth measurement period MRK. It should be understood, however, that the disclosure is not limited thereto. For example, according to embodiments, the processor 800 may calculate the polynomial correction function DPL based on the amplitudes of four or more measurement segments.

According to an embodiment, the processor 800 generates the second pulse wave signal PPG2 by interpolating the pulse waveform removed in the abnormal measurement segment of the first pulse wave signal PPG1. For example, the processor 800 may generate the second pulse wave signal PPG2 by correcting the signal of the abnormal measurement segment in the first pulse wave signal PPG1. Accordingly, when the display device 1 calculates the blood pressure based on the pulse wave signal, an accurate blood pressure can be calculated by correcting the abnormal pulse waveform.

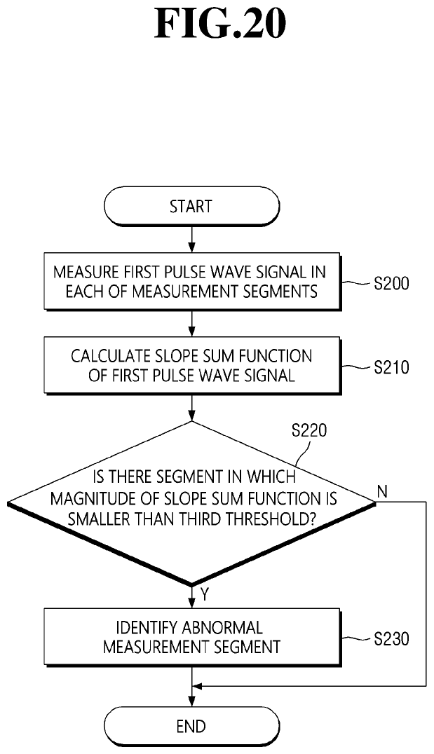

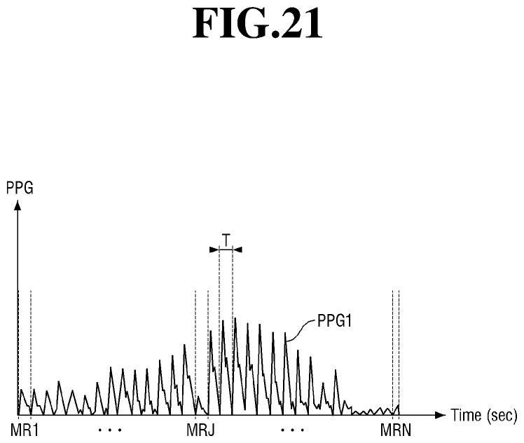

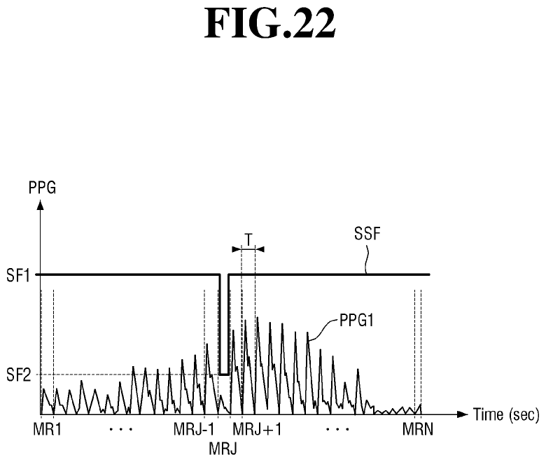

FIG. 20 is a flowchart illustrating a method of identifying an abnormal measurement segment by a display device according to an embodiment of the disclosure. FIG. 21 is a graph showing a pulse wave signal according to an embodiment of the disclosure. FIG. 22 is a graph showing a pulse wave signal and a slope sum function according to an embodiment of the disclosure. Hereinafter, a method of identifying an abnormal measurement segment by the display device will be described with reference to FIGS. 20 to 22.

Initially, referring to FIG. 20, a pulse wave sensing circuit 50 measures a first pulse wave signal PPG1 in each measurement segment (operation S200). As described above, the first pulse wave signal PPG1 has one cycle in each of the first to n^{th} measurement segments MR1 to MRN, and the first pulse wave signal PPG1 includes waveforms having a plurality of amplitudes in each of the first to n^{th} measurement segments MR1 to MRN. The first pulse wave signal PPG1 is substantially identical to the first pulse wave signal PPG1 of FIG. 7. Thus, for convenience of explanation, a redundant description thereof will be omitted.

Subsequently, a slope sum function SSF of the first pulse wave signal PPG1 is calculated (operation S210).

Referring to FIGS. 21 and 22, the slope sum function SSF may be defined as in Equation 1 below:

$${SSF}_{i} = {\sum }_{k = i - w}^{j} Δ {u}_{k} , Δ {u}_{k} = \{ \begin{matrix}Δ {x}_{k} & : & Δ {x}_{k} > 0 \\ 0 & : & Δ {x}_{k} \leq 0\end{matrix} \qquad [ Equation 1 ]$$

where x denotes sample values included in the first pulse wave signal PPG1, w denotes an increase in one measurement segment, i denotes an integer greater than 1+w and less than N, and SSFi denotes sample values included in the slope sum function SSF. According to an embodiment of the disclosure, the width w of the measurement segments may be substantially equal to the length of the increasing duration of the first pulse wave signal PPG1 from the start point of the measurement segment to the first amplitude PP1.

As expressed in Equation 1, when the sample value of the first pulse wave signal PPG1 increases during one measurement segment in the first pulse wave signal PPG1, the slope sum function SSF may be generated by summing the degrees of increasing.

Therefore, as shown in FIG. 22, when the first pulse wave signal PPG1 decreases or maintains a constant value, the value of the slope sum function SSF is held. According to embodiments, the slope sum function SSF may increase only when the first pulse wave signal PPG1 increases. For example, a first value SF1 generated in the slope sum function SSF may be equal to the first amplitude PP1 of the measured first pulse wave signal PPG1.

On the other hand, since the first pulse wave signal PPG1 includes the abnormal signal in the abnormal measurement segment, the value of the slope sum function SSF may be different from the first value SF1 in the abnormal measurement segment. For example, in the jth measurement segment MRJ, where j is a positive integer, the slope sum function SSF may have a second value SF2. The second value SF2 may be equal to the second amplitude PP2 of the first pulse wave signal PPG1 in the jth measurement segment MRJ. In this case, the second value SF2 may be smaller than the first value SF1.

Subsequently, if there is a segment in which the magnitude of the slope sum function SSF is smaller than the third threshold value (yes in operation S220), the processor 800 may identify the segment as an abnormal measurement segment (operation S230).

The processor 800 may compare the magnitude of the slope sum function SSF with the third threshold value in each of the first to n^{th} measurement segments MR1 to MRN. For example, the processor 800 may determine that the first value SF1 of the slope sum function SSF is greater than the threshold value in the first to (j−1)^{th} measurement segments MR(J−1) and the (j+1)^{th} to n^{th} measurement segments. If so, according to an embodiment, the processor 800 does not identify any abnormal measurement segment. In addition, the processor 800 may determine that the second value SF2 of the slope sum function SSF is smaller than the third threshold value in the jth measurement segment MRJ. If so, the processor 800 may identify the jth measurement segment MRJ as an abnormal segment.

In a method of measuring blood pressure of the display device 1 according to an embodiment, the slope sum function SSF is generated based only on the degrees by which the measured first pulse wave signal PPG1 increases, and thus, the abnormal measurement segment in the first pulse wave signal PPG1 may be accurately detected.

FIG. 23 is a flowchart illustrating a method of calculating blood pressure according to an embodiment of the disclosure. FIG. 24 is a graph showing a waveform of a peak detection signal according to an embodiment of the disclosure. A method of calculating blood pressure based on a third pulse wave signal PPG3 will be described with reference to FIGS. 23 and 24.

Initially, referring to FIGS. 23 and 24, a processor 800 generates a peak detection signal PPS (operation ST1).

The processor 800 may calculate the amplitude at every cycle T of the third pulse wave signal PPG3. In addition, the processor 800 may generate a peak detection signal PPS having the magnitudes of the third pulse wave signal PPG3 based on the amplitude at each cycle T of the third pulse wave signal PPG3. For example, the peak detection signal PPS is defined as a signal associated with the amplitude of each cycle of the third pulse wave signal PPG3. For example, the peak detection signal PPS may be defined as a signal associated with the peak value of each cycle of the third pulse wave signal PPG3. For example, the third pulse wave signal PPG3 may have at least one amplitude. The processor 800 may generate the peak detection signal PPS including points each corresponding to the amplitude of the respective cycle T of the third pulse wave signal PPG3. For example, the generated peak detection signal PPS may be a signal having an amplitude according to pressure.

Subsequently, the processor 800 determines whether a pressure value corresponding to the peak value PK of the peak detection signal PPS can be calculated (operation ST2). If there is a peak of the peak detection signal PPS, the processor 800 may calculate a pressure value corresponding to the peak value PK of the peak detection signal PPS.

Subsequently, the processor 800 calculates systolic blood pressure SBP, diastolic blood pressure DBP, etc. based on the peak value PK of the peak detection signal PPS (operation ST3), and yields the blood pressure information (operation ST4).

The processor 800 may calculate the diastolic blood pressure DBP lower than the pressure value, the systolic blood pressure SBP higher than the pressure value, and the average blood pressure based on the pressure value. For example, the processor 800 may calculate pressure values of about 60% to about 80% of the peak value PK. A pressure value smaller than the pressure value corresponding to the peak value PK among the pressure values may be calculated as a first pressure value PR1. In addition, the processor 800 may calculate the first pressure value PR1 as the diastolic blood pressure DBP. In addition, the processor 800 may calculate a pressure value greater than the pressure value corresponding to the peak value PK among the pressure values as a second pressure value PR2. In addition, the processor 800 may calculate the second pressure value PR2 as the systolic blood pressure SBP.

According to an embodiment, since the third pulse wave signal PPG3 oscillates with every heartbeat cycle, the third pulse wave signal PPG3 can reflect a change in the blood pressure according to the heartbeat. As a result, the display device 1 can yield the blood pressure information accurately based on the third pulse wave signal PPG3 according to embodiments of the disclosure.

According to an embodiment, the display device identifies an abnormal measurement segment and removes and interpolates the pulse wave signal in the abnormal segment. In addition, since the display device calculates the blood pressure based on the interpolated pulse wave signal, blood pressure may be accurately measured.

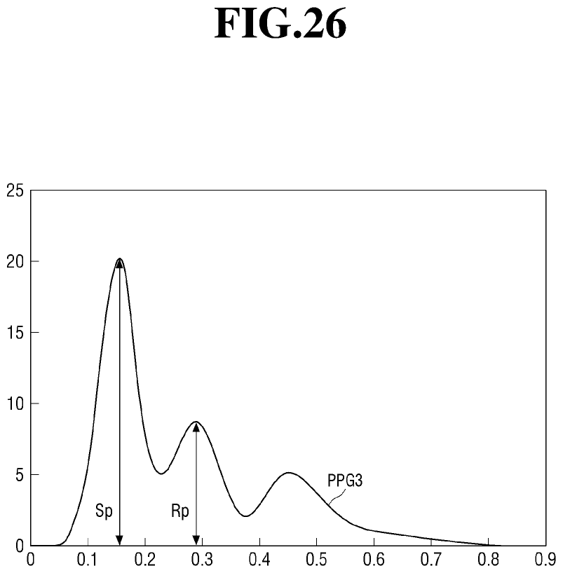

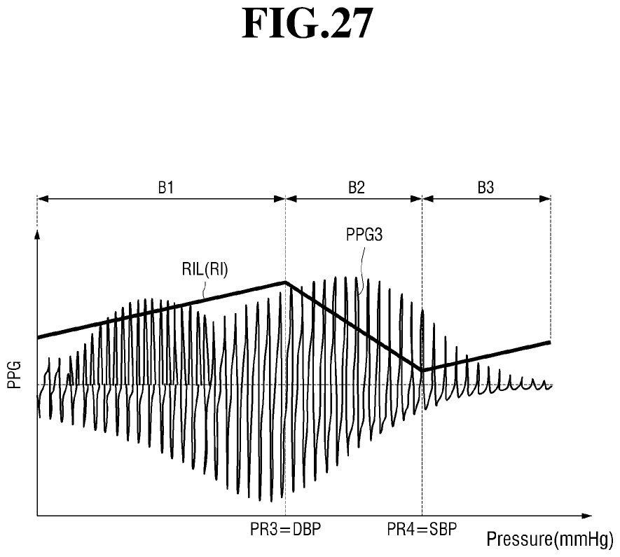

FIG. 25 is a flowchart illustrating a method of calculating blood pressure according to an embodiment of the disclosure. FIG. 26 is a graph showing a waveform of one cycle of a pulse wave signal according to an embodiment of the disclosure. FIG. 27 is a graph showing a pulse wave signal and a reflected pulse wave ratio according to an embodiment of the disclosure. Hereinafter, a method of calculating blood pressure by a display device based on the reflected pulse wave ratio RI will be described with reference to FIGS. 25 to 27.

Referring first to FIG. 25, a reflected pulse wave ratio RI is calculated for every cycle of a third pulse wave signal PPG3 (operation S610).

Referring further to FIG. 27, in order to calculate the reflected pulse wave ratio RI, the processor 800 divides the wave cycles of a third pulse wave signal PPG3 into a wave by heartbeat and a reflected wave of a blood vessel by the durations in which they are generated. For example, one cycle of the third pulse wave signal PPG3 may include a plurality of waveforms having different amplitudes. A peak value of a waveform having the greatest amplitude among a plurality of waveforms may be defined as a systolic pulse wave value, and a peak value of the waveform having the second greatest amplitude among the plurality of waveforms may be defined as a reflected pulse wave value. The systolic pulse wave value is denoted by Sp, reflected pulse wave value is denoted by Rp, and the reflected pulse wave ratio is denoted by RI. The reflected pulse wave ratio RI may be calculated by Equation 2 below:

$$RI = \frac{R p}{S p} \qquad [ Equation 2 ]$$

where the systolic pulse wave value Sp may have the same value as the main amplitude in each of the first to n^{th} measurement segments. The peak value Rp of the waveform having the second greatest amplitude may be substantially equal to the sub-amplitude in each of the first to n^{th} measurement segments.

According to embodiments, the processor 800 may calculate the peak value Sp of the waveform having the greatest amplitude among a plurality of waveforms included in one cycle of the third pulse wave signal PPG3. In addition, the processor 800 may calculate the peak value Rp of the waveform having the second greatest amplitude among the plurality of waveforms included in one cycle of the third pulse wave signal PPG3. In addition, the processor 800 may calculate the reflected pulse wave ratio RI based on the systolic pulse wave value Sp and the reflected pulse wave value Rp.

Second, the processor 800 determines whether a second duration B2 of the reflected pulse wave ratio RI can be calculated (operation S620). The processor 800 sequentially stores the detection results of the reflected pulse wave ratio RI as the systolic pulse wave value, and analyzes the stored reflected pulse wave ratio RI. The processor 800 may analyze a change in the magnitude of the reflected pulse wave ratio data RIL(RI) by continuously converting the magnitude change of the reflected pulse wave ratio RI into data.

The reflected pulse wave ratio RI includes a first duration B1 in which the reflected pulse wave ratio RI fluctuates within a first range, a second duration B2 in which the reflected pulse wave ratio RI fluctuates within a second range, and a third duration B3 in which reflected pulse wave ratio RI fluctuates within a third range. For example, the processor 800 may analyze the reflected pulse wave ratio signal RIL, to analyze the first duration B1 in which the reflected pulse wave ratio RI is saturated and thus is less variably changed within a predetermined range, the second duration B2 in which the reflected pulse wave ratio RI rapidly decreases or increases out of the predetermined range within a predetermined period, and the third duration B3 in which the reflected pulse wave ratio (RI) is saturated again and thus is less variably changed within the predetermined range after it rapidly decreases or increases.

The width of the first range and the width of the third range may be smaller than the width of the second range. In addition, the slope of the second duration B2 of the reflected pulse wave ratio RI may be greater than the slope of the reflected pulse wave ratio RI in the first duration B1 and the slope of the reflected pulse wave ratio RI in the third duration B3.

Finally, the processor 800 calculates a systolic blood pressure SBP, a diastolic blood pressure DBP, etc. based on the reflected pulse wave ratio RI (operation S630), and yields the blood pressure information (operation S640).

The processor 800 may analyze the reflected pulse wave ratio RI to detect the start point of the second duration B2. Then, the processor 800 may calculate a third pressure value PR3 corresponding to the third pulse wave signal PPG3 at the start point of the second duration B2. The processor 800 may calculate the third pressure value PR3 as the diastolic blood pressure DBP. In addition, the processor 800 may analyze the reflected pulse wave ratio RI to detect the start point of the third duration B3 after the second duration B2. Then, the processor 800 may calculate a fourth pressure value PR4 corresponding to the third pulse wave signal PPG3 at the start point of the third duration B3. The processor 800 may calculate the fourth pressure value PR4 as the systolic blood pressure SBP.

As is traditional in the field of the present disclosure, embodiments are described, and illustrated in the drawings, in terms of functional blocks, units and/or modules. Those skilled in the art will appreciate that these blocks, units and/or modules are physically implemented by electronic (or optical) circuits such as logic circuits, discrete components, microprocessors, hard-wired circuits, memory elements, wiring connections, etc., which may be formed using semiconductor-based fabrication techniques or other manufacturing technologies. In the case of the blocks, units and/or modules being implemented by microprocessors or similar, they may be programmed using software (e.g., microcode) to perform various functions discussed herein and may optionally be driven by firmware and/or software. Alternatively, each block, unit and/or module may be implemented by dedicated hardware, or as a combination of dedicated hardware to perform some functions and a processor (e.g., one or more programmed microprocessors and associated circuitry) to perform other functions.

According to an embodiment, the display device identifies an abnormal measurement segment and removes and interpolates the pulse wave signal in the abnormal segment. In addition, since the display device calculates the blood pressure based on the interpolated pulse wave signal, blood pressure may be accurately detected.

While the present disclosure has been particularly shown and described with reference to embodiments thereof, it will be understood by those of ordinary skill in the art that various changes in form and detail may be made therein without departing from the spirit and scope of the present invention as defined by the following claims.

What is claimed is:

1. A display device, comprising:
a display panel comprising a plurality of pixels;
a pressure sensor configured to sense a pressure applied from outside of the display device;
a photosensor configured to detect a light; and
a processor configured to receive a pressure signal sensed by the pressure sensor in each of a plurality of measurement segments, and a first pulse wave signal sensed by the photosensor in each of the plurality of measurement segments,
wherein the processor is further configured to:
identify at least one of the measurement segments as an abnormal measurement segment when a magnitude of the pressure signal sensed in the at least one of the measurement segments does not lie in a predetermined first threshold range;
remove a pulse waveform from the first pulse wave signal in the abnormal measurement segment;
generate a second pulse wave signal by interpolating the pulse waveform;
calculate a third pulse wave signal having an amplitude of the second pulse wave signal based on the pressure signal sensed by the pressure sensor and the second pulse wave signal; and
calculate a blood pressure based on the third pulse wave signal.

2. The display device of claim 1,
wherein the first threshold range comprises an upper limit pressure, a lower limit pressure, and a pressure width, and the pressure width is within 10 mmHg.

3. The display device of claim 2,
wherein the upper limit pressure and the lower limit pressure gradually increase in the plurality of measurement segments.

4. The display device of claim 1,
wherein the processor receives an additional pressure signal from the pressure sensor in each of the plurality of measurement segments when the magnitude of the pressure signal sensed in the at least one of the measurement segments does not lie within a predetermined second threshold range.

5. The display device of claim 4,
wherein the processor receives the first pulse wave signal from the photosensor a plurality of times.

6. The display device of claim 5,
wherein a pressure width of the second threshold range is greater than a pressure width of the first threshold range.

7. The display device of claim 1,
wherein the plurality of measurement segments comprises first to n^{th} measurement segments, and the first pulse wave signal has one cycle in each of the first to n^{th} measurement segments, and
wherein n is a positive integer.

8. The display device of claim 7,
wherein when a kth measurement segment is the abnormal measurement segment, the processor generates the second pulse wave signal by calculating an average value of an amplitude of a (k−1)^{th} measurement segment and an amplitude of a (k+1)^{th} measurement segment as an amplitude of the pulse waveform, and
wherein k is a positive integer.

9. The display device of claim 8,
wherein the processor generates a peak detection signal based on an amplitude corresponding to a peak of each cycle of the third pulse wave signal.

10. The display device of claim 9,
wherein the processor calculates a peak value of the peak detection signal and a pressure value corresponding to the peak value of the peak detection signal, a diastolic blood pressure lower than the pressure value, a systolic blood pressure higher than the pressure value, and an average blood pressure depending on the pressure value.

11. The display device of claim 10,
wherein the processor calculates the pressure value corresponding to the peak value as the average blood pressure.

12. The display device of claim 11,
wherein a first pressure value smaller than a pressure value of about 60% to about 80% of the peak value in the peak detection signal and a second pressure value greater than the pressure value are calculated, and the first pressure value is calculated as the diastolic blood pressure and the second pressure value is calculated as the systolic blood pressure.

13. The display device of claim 7,
wherein each cycle of the first pulse wave signal comprises a plurality of waveforms having different amplitudes, and a kth measurement segment is the abnormal measurement segment,
wherein the processor generates the second pulse wave signal by calculating an average value of a pulse waveform of a (k−1)^{th} measurement segment and a pulse waveform of a (k+1)^{th} measurement segment as the pulse waveform, and
wherein k is a positive integer.

14. The display device of claim 13,
wherein one cycle of the third pulse wave signal comprises a plurality of waveforms having different amplitudes,
wherein a reflected pulse wave ratio is calculated by:
$$RI = \frac{{R}_{P}}{{S}_{P}}$$
wherein a peak value of a first waveform among the plurality of waveforms is defined as a systolic pulse wave value, and a peak value of a second waveform among the plurality of waveforms is defined as a reflected pulse wave value, and
wherein the systolic pulse wave value is denoted by Sp, the reflected pulse wave value is denoted by Rp, and the reflected pulse wave ratio is denoted by RI.

15. The display device of claim 14,
wherein the reflected pulse wave ratio comprises a first duration in which the reflected pulse wave ratio fluctuates within a first range, a second duration in which the reflected pulse wave ratio fluctuates within a second range, and a third duration in which the reflected pulse wave ratio fluctuates within a third range, and
wherein a width of the first range and a width of the third range are less than a width of the second range.

16. The display device of claim 15, wherein the processor is further configured to:
analyze the reflected pulse wave ratio to detect a start point of the second duration;
calculate a first pressure value corresponding to the first pulse wave signal at a start time of the second duration;
set the first pressure value as a diastolic blood pressure;
calculate a second pressure value corresponding to the first pulse wave signal at a start time of the third duration; and
calculate the second pressure value as a systolic blood pressure.

17. A method of calculating a blood pressure by a display device, comprising:
sensing, by a pressure sensor configured to sense a pressure applied from outside of the display device, a pressure signal in each of first to n^{th} measurement segments, and when a magnitude of the pressure signal sensed in one of the measurement segments does not lie within a predetermined first threshold range, identifying the measurement segment as an abnormal segment,
wherein n is a positive integer;
sensing, by a photosensor configured to sense light, a first pulse wave signal having one cycle in each of the first to n^{th} measurement segments, and removing a pulse waveform of the first pulse wave signal in the abnormal measurement segment;
generating a second pulse wave signal by interpolating the pulse waveform;
calculating a third pulse wave signal having an amplitude of the second pulse wave signal based on the pressure signal and the second pulse wave signal; and
calculating the blood pressure based on the third pulse wave signal and indicating blood pressure information on a display panel of the display device.

18. The method of claim 17,
wherein generating the second pulse wave signal by interpolating the pulse waveform comprises:
when a kth measurement segment is the abnormal measurement segment, generating the second pulse wave signal by calculating an average value of an amplitude of a (k−1)^{th} measurement segment and an amplitude of a (k+1)^{th} measurement segment as an amplitude of the pulse waveform,
wherein k is a positive integer.

19. The method of claim 18,
wherein calculating the blood pressure based on the third pulse wave signal and indicating the blood pressure information on the display panel comprises:
generating a peak detection signal based on an amplitude corresponding to a peak of each cycle of the third pulse wave signal;
calculating a peak value of the peak detection signal and a pressure value corresponding to the peak value of the peak detection signal; and
calculating a diastolic blood pressure lower than the pressure value, a systolic blood pressure higher than the pressure value, and an average blood pressure depending on the pressure value.

20. The method of claim 19,
wherein the pressure value corresponding to the peak value is calculated as the average blood pressure.
